(19) United States
(12) Patent Application Publication
(10) Pub. No.: US 2003/0181407 A1
(43) Pub. Date: Sep. 25, 2003

(54) Steatosis-modulating factors and uses thereof

(76) Inventors: Marie-France Palin, Waterville (CA); Candido Pomar, Fleurimont (CA); Claude Gariepy, St-Hilaire (CA)

Correspondence Address: WOOD, PHILLIPS, KATZ, CLARK & MORTIMER, 500 W. MADISON STREET, SUITE 3800, CHICAGO, IL, 60661, US

(21) Appl. No.: 10/257,826

(22) PCT Filed: Apr. 12, 2001
(86) PCT No.: PCT/CA01/00509

(51) Int. Cl. A61K 48/00; C12Q 1/68

(52) U.S. Cl. 514/44; 435/6

(57) ABSTRACT

The present invention relates to a method of modulating the muscular steatosis-modulating factors (MSMF). The determination of concentrations of the MSMF is described for the establishment of the steatotic state in individuals. Also, is disclosed a method of selecting individuals to serve as founders of animal lineages. The present method involved too the treatment of human and animals with agonists or antagonists of MSMF depending of the effects desired.

### BACKGROUND OF THE INVENTION

(a) Field of the Invention

The invention relates to a measurement of the level of muscular steatosis-modulating factor (MSMF) in human or animal. The method is performed by measuring level of MSMF in a biological sample, and then screening individual having normal and abnormal level of MSMF.

(b) Description of Prior Art

Mammalian skeletal muscle normally undergoes a reparative process after oxidative stress or traumatic injury. The process of skeletal muscle repair is actually a series of discrete overlapping events, which can be segregated into trauma, tissue degeneration, inflammation, phagocytosis, angiogenesis, stem cell activation, migration of the stem cells to the site of injury, proliferation of undifferentiated stem cells, re-innervation, differentiation of the stem cells, and remodeling of the tissue.

The early restored muscle tissues approximate embryonic-like satellite cells containing centrally located nuclei and lies adjacent to mature myofibers containing peripherally located nuclei. Unfortunately, restoration of physiological function may be compromised due to the increased proliferative nature of the surrounding connective tissues, eventually forming non-functional scar tissue.

Research in other areas has indicated that various factors such as platelet derived growth factor (PDGF), chicken muscle growth factor (CMGF), epidermal growth factor (EGF), sciatic nerve extract, insulin, and somatomedins stimulate a mitogenic or proliferative response in cultured muscle cells. This response should be contrasted with a myogenic response that does not induce myogenic lineage commitment of uncommitted stem cells, but instead induces the lineage commitment of the stem cells.

Three growth factors, insulin and insulin-like growth factors, namely insulin-like growth factor-I (IGF-I), also called somatomedin-C, insulin-like growth factor-II (IGF-II), also called myogenic stimulating activity, have been shown to be potent stimulators of skeletal muscle cell growth and differentiation in cultured myosatellite cells and myogenic lineage-committed stem cells by Ewton and Florini, Dev. Biol. 83:31-39 (1981); Florini et al., J. Biol. Chem. 261:16509-16515 (1986); Sejersen et al., Proc. Natl. Acad. Sci. 83:6844-6848 (1986).

Several in vivo studies have employed basic-fibroblast growth factor (FGF-2) also named FGF-2, transforming growth factor beta (TGF-beta), and epidermal growth factor (EGF) to stimulate internal wound healing. Buckley et al., Proc. Natl. Acad. Sci. 82:7340-7344 (1985); and Roberts et al., Proc. Natl. Acad. Sci. 83:4167-4171 (1986) noted that administration of FGF-2, TGF-beta, and EGF appeared to promote proliferation of connective tissue elements to form scar tissue and thus aid in wound healing of mammalian skeletal muscle.

In vitro studies have demonstrated the influence of other growth factors on the resultant phenotypic expression in myogenic cultures. For example, Hauschka (Lim and Hauschka, J. Cell Biol. 98:739-747 (1984); and Olwin and Hauschka, Biochemistry 25:3487-3492 (1986)) and co-workers have reported that acidic-fibroblast growth factor (aFGF) and basic-fibroblast growth factor (FGF-2) play a dual role in stimulating myoblast proliferation while directly repressing terminal differentiation, as described by Linkhart et al., Dev. Biol. 86:19-30 (1981).

Unfortunately, the administration of growth factors that inhibit terminal myogenic differentiation, promote myoblast proliferation, and promote fibroblast proliferation and differentiation as a method to promote muscle repair appears to cause an increase in connective tissue scar formation. In muscle, increased scar formation creates decreased physiological function. A decrease in connective tissue scar formation with, a compensatory increase in skeletal muscle mass plus revascularization and re-innervation of the tissues is necessary for the restoration of physiological function.

Obesity has been declared a public health hazard by the National Institutes of Health. To combat this health problem, both prophylactic and therapeutic approaches are necessary. For prophylactic purposes, it would be useful to be able to predict and measure a person's propensity or susceptibility to obesity for therapeutic purposes, a means for interfering with the development or differentiation of adipocytes (fat cells) would be of great benefit. Furthermore, as a broader preventative approach to obesity, the ability to limit the fat content of food mammals would be of great importance. None of these desired objectives has been achieved. A weight reduction program cannot efficiently control early-onset obesity once the obesity is apparent. Therefore, a means for early detection of early-onset obesity is imperative for its prevention.

It is held that excessive ingestion of fat and carbohydrate induces obesity and hyperlipidemia and even develops hypertension and arteriosclerosis ultimately. The desirability of repressing the absorption of fat and carbohydrate and diminishing the accumulation of fat has, therefore, been finding enthusiastic recognition.

Infants, on exposure to excessive ingestion of nutriments, suffer increase of adipocytes and assume the state that may well be called potential obesity. For this reason, it has been reported that the repression of the increase of the number of adipocytes particularly in infants results directly in the prevention of the obesity and the cardiovascular diseases which may well be called complications of obesity in children and consequently in adults.

For the therapy of obesity and hyperlipidemia, such measures as limitation of meal, ingestion of diet food (such as, for example, fibers), and even administration of various medicines have been in vogue. The medicines now in popular use include dextran sulfate which enhances the lipoprotein lipase activity in blood, Nicomol™ that inhibits absorption of lipid, especially cholesterol, and Clofibrate™ and Pravastatin™ which are agents for improving metabolism of lipid, for example.

Unfortunately, the limitation of meal is an agony for persons obliged to pursue this exercise and the administration of such medicines as mentioned above possibly entrains side effects.

Replacement of myofibres by adipose cells, usually with no decrease in muscle volume is defined as muscular steatosis.

Reports concerning muscular steatosis (MSt) in animals is alternatively named progressive primary myopathy, pseudohypertrophic atrophy, lipomatous pseudohypertrophy, interstitial lipomatosis, lipomatous muscular dystrophy, myosclerosis, and hypoplasia or atrophia lipomatosa. MSt is typically found in otherwise healthy cattle and pigs but it also occurs in dogs, sheep, fish, birds and human. Cattle with MSt sometimes have an abnormal gait with hind feet knuckled over and erratic hind limb movements. Affected animals stand normally, but sway or stagger when blindfolded. Lesions are usually bilaterally symmetrical and may appear almost anywhere in the carcass, although longissimus dorsi and hind limb muscles are most frequently affected. Myofibres in affected areas may lack transverse striations and may be fragmented or vacuolated. Remaining myofibres may be hypertrophied, possibly a compensatory mechanism, or atrophied with an increase in number of nuclei. An important feature is that there is inflammatory cells usually invade no evidence of myofibre regeneration in MSt. Areas of MSt. Proliferation, or replacement by adipose cells is a common finding in many myopathies, especially terminal cases, and does not necessarily indicate MSt.

Muscles of meat animals, especially at market weight, contain large numbers of adipose cells that play a major role in the determination of meat quality. Since adipose tissue is normally found intramuscularly, MSt must be viewed in the context of normal intramuscular adipose tissue accumulation. It might be difficult to distinguish between minimal MSt and maximal accumulation of adipose cells in muscles showing a normal reduction in apparent number of myofibres.

Effects of denervation are very variable but extramuscular denervation usually results in atrophy rather than MSt. MSt probably results from a combination of myofibre damage, motor denervation, autonomic re-innervation and positive caloric balance occurring as a result of intramuscular denervation in a growing animal. The normal intramuscular adipose tissue pattern is retained in areas of MSt, and fatty acid composition is similar to subcutaneous fat with a high amount of unsaturated fatty acids. In naturally occurring MSt, denervation alone would be unlikely to cause a major lesion because of the efficiency of collateral re-innervation by surviving neurons.

It is possible that if intramuscular denervation had occurred in conjunction with muscle rupture, MSt rather than fibrosis would be the result. It may be no coincidence that MSt is typically observed in heavily muscled meat animals in locations (loin and hind limb) that might be damaged by muscular exertion during locomotion or mating. MSt in one area of a muscle might predispose adjacent areas to trauma on subsequent exertion, thus accounting for the considerable tracts of MSt that may occur. The alternative hypothesis to self-inflicted muscle damage is that MSt is due to a defective development of vascular tissues. Although blood vessels with abnormally thick walls and surrounded by connective tissues may be observed in naturally occurring MSt, this might also be related to muscle damage.

With classical histological techniques, intermyofibrillar lipid droplets were distinguished from interstitial granules (mitochondria), and both were found to be more abundant in “dark” myofibres. Lipid staining droplets occur in bovine fetuses and in the atrophic muscles of steers on a submaintenance diet. The abnormal accumulation of lipid droplets may occur in myofibres either as a non-specific response to myofibre degeneration or through a defect in long chain fatty acid utilization. It is possible that lipid accumulation myopathy is an initial cause of MSt.

Traverse muscle sections in myopathic conditions, polygonal cells resembling myofibres have a glassy appearance, are uniformly sudanophilic and are not exhibiting any reaction for beta-hydroxybutyric dehydrogenase, as are adjacent red myofibres. With light microscopy, myofibrillar disruption, lipid infiltration and loss of birefringence can be observed within porcine myofibres. Subsequent electron microscopy shows that changes can be due to dissociation of groups of myofibrils, contraction of sarcomeres, loss of density in the A band and fragmentation of myofibrils. Lipid infiltration is confirmed, and it is also observed that the sarcolemma is detached and thickened and that mitochondria have wasted matrices and fragmented cristae.

Human lipid accumulation myopathies most often involve the red or type 1 myofibres is no coincidence that aerobic metabolism, the typical function of red myofibres, is deficient in SS-lineage pigs and that red myofibres are more easily damaged by ischaemia.

Different molecules, growth hormones, growth factors, lipids and other have been studied in association with the adipogenesis and myogenesis mechanisms. Among those factors, there is considered acidic and basic fibroblast growth factor (aFGF, FGF-2), transforming growth factor-beta and -alpha (TGF-α and TGF-α), adipocyte differentiating related protein (ADRP), epidermal growth factor (EGF), insulin like growth factor 1 and 2 (IGF-1 and IGF-2), IGF-1 receptor and IGF-2 receptor, platelet derived growth factor-alpha and -beta (PDGF-α and PDGF-β), leptin, and lipoprotein lipase (LPL).

Epidermal growth factor (EGF) is a 6-kDa molecular weight polypeptide found in high concentrations in the submaxillary glands and at lower levels in the circulation. EGF affects the proliferation and the maintenance of functional properties of various mammalian cells in vitro (13-14). Animal experiments involving either injection of EGF, injection of antibodies specific for EGF, or removal of the major source of EGF by sialoadenectomy, have shown that EGF played a physiological role on the maintenance of several tissue functions in vivo.

IGF-I and IGF-II are growth factors that have related amino acid sequence and structure, with each polypeptide having a molecular weight of approximately 7.5 kilodaltons (kDa). IGF-I mediates the major effects of growth hormone, and thus is the primary mediator of growth after birth. IGF-I has also been implicated in the actions of various other growth factors, since treatment of cells with such growth factors leads to increased production of IGF-I. In contrast, IGF-II is believed to have a major role in fetal growth. Both IGF-I and IGF-II have insulin-like activities (hence their names) and are mitogenic (stimulate cell division) and/or are trophic (promote recovery/survival) for cells in neural, muscular, reproductive, skeletal and other tissues.

Unlike most growth factors, IGFs are present in substantial quantity in the circulation, but only a very small fraction of this IGF is free in the circulation or in other body fluids. Most circulating IGF is bound to the IGF-binding protein IGFBP-3. IGF-I may be measured in blood serum to diagnose abnormal growth-related conditions, e.g., pituitary gigantism, acromegaly, dwarfism, various growth hormone deficiencies, and the like. Although IGF-I is produced in many tissues, most circulating IGF-I is believed to be synthesized in the liver.

Almost all IGF circulates in a non-covalently associated ternary complex composed of IGF-I or IGF-II, IGFBP-3, and a larger protein subunit termed the acid labile subunit (ALS). The IGF/IGFBP-3/ALS ternary complex is composed of equimolar amounts of each of the three components. ALS has no direct IGF binding activity and appears to bind only to the IGF/IGFBP-3 binary complex. The IGF/IGFBP-3/ALS ternary complex has a molecular weight of approximately 150 kDa. This ternary complex is thought to function in the circulation “as a reservoir and a buffer for IGF-I and IGF-II preventing rapid changes in the concentration of free IGF.

One other of these, the Insulin-Like Growth Factor-I Receptor (IGF-IR) is a member of the tyrosine kinase family of signal transducing molecules. The IGF-IR is activated by the ligands IGF-I, IGF-II and insulin at supra-physiological concentrations, and plays an important role in the development, growth, and survival of normal cells. Over-expression of the IGF-IR leads to the transformation of fibroblasts and conversely, IGF-IR null fibroblasts are refractory to transformation by a number of oncogenes. Fibroblasts from IGF-IR null mice have been used to demonstrate a requirement for the IGF-IR in transformation, and also to map domains in the receptor essential for the proliferative and transformation function of the IGF-IR. Specifically, the C-terminal region of the IGF-IR is required for the transformation function. Receptors, which are truncated at amino acid 1229 fail to transform fibroblasts derived from IGF-IR, null mice, but retain full proliferative activity.

PDGF is considered to be a principal growth-regulatory molecule responsible for smooth muscle cell proliferation. For instance, PDGF, as measured by mRNA analysis as well as in situ staining using an antibody against PDGF, was found within macrophages of all stages of lesion development in both human and nonhuman primate atherosclerosis. PDGF was found in both non-foam cells and lipid rich macrophage foam cells. These data are consistent with PDGF playing a critical role in the atherosclerosis disease process. In addition, analysis of advanced human lesions examined by atherectomy catheter indicates that atherosclerotic and restenotic lesions contain high levels of PDGF as measured by in situ hybridization.

Human transforming growth factor-beta (TGF-beta) has been isolated from human blood platelets and placenta and purified to essential homogeneity using sequential gel filtration cation-exchange chromatography and high performance liquid chromatography. The purified protein has been characterized as having a molecular weight of 25,000 daltons and composed of 2 sub-units of 12,500 daltons each held together by disulfide bonds. The molecular weight, sub-unit structure and amino acid composition of the purified protein differed from that of platelet derived growth factor.

TGF-beta has also been purified from platelets or conditioned media utilizing acid ethanol extraction, cation-exchange separation on the extract, and hydrophobic separations on the active fractions to obtain a homogenous preparation. The purified product is said to be useful in wound healing and tissue repair.

TGF-beta has also been prepared utilizing recombinant DNA, wherein the cloned human gene coding for TGF-beta was inserted into eukaryotic cell lines for expression. The protein product was said to be useful in promoting anchorage-dependent or independent growth in cell culture.

The idea that FGF-2 antagonists may have useful medicinal applications is not new. FGF-2 is now known to play a key role in the development of smooth-muscle cell lesions following vascular injury. Overexpression of FGF-2 (and other members of the FGF family) is correlated with many malignant disorders (Takahashi et al. (1990) Proc. Natl. Acad. Sci. U.S.A. 87:5710). Neutralizing anti-FGF-2 antibodies have been found to suppress solid tumor growth in vivo by inhibiting tumor-linked angiogenesis (Hori et al. (1991) Cancer Res. 51:6180). Notable in this regard is the recent therapeutic examination of suramin, a polysulfated naphthalene derivative with known antiprotozoal activity, as an anti-tumor agent. Suramin is believed to inhibit the activity of FGF-2 through binding in the polyanion binding site and disrupting interaction of the growth factor with its receptor (Middaugh et al. (1992) Biochemistry 31:9016). In addition to having a number of undesirable side effects and substantial toxicity, suramin is known to interact with several other heparin-binding growth factors, which makes linking of its beneficial therapeutic effects to specific drug-protein interactions difficult. Anti-angiogenic properties of certain heparin preparations have also been observed (Folkman et al. (1983) Science 221:719; Crum et al. (1985) Science 230:1375) and these effects are probably based at least in part on their ability to interfere with FGF-2 signaling. While the specific heparin fraction that contributes to FGF-2 binding is now partially elucidated, a typical heparin preparation is heterogeneous with respect to size, degree of sulfation and iduronic acid content. Additionally, heparin also affects many enzymes and growth factors. Basic FGF is thought to regulate myogenesis during muscle development and regeneration in vivo. The increase percentage of muscle fibers containing the donor gene produced by the addition of FGF-2 may seem surprising since FGF-2 was reported to inhibit differentiation of myoblasts in vitro. Basic FGF is, however, one of many growth factors, which are liberated following muscle damage. These factors, all together, certainly increase myoblast proliferation and eventually muscle repairs. It has been also observed that following two days incubation with FGF-2 of primary myoblast cultures, myoblast fusion occurred within a few days after removal of FGF-2. The inhibition by FGF-2 on myoblast fusion is therefore not irreversible. Basic FGF is already at an increased level in mdx muscle, therefore it is not surprising that direct intramuscular injection did not increase the fusion of the donor myoblasts with the host fibers. In fact, FGF-2 injected directly in the muscle probably stimulates the proliferation of the host as well as the donor myoblasts and therefore does not favor the donor myoblasts. On the contrary, preliminary stimulation by FGF-2 of the donor myoblasts in culture may favor these myoblasts to proliferate more and eventually participate more to muscle regeneration than the host myoblasts. Although FGF-2 stimulates the fibroblasts, a result, which could pose an inconvenience to primary myoblast cultures, the 48 hours incubation of myoblast primary culture with FGF-2, did not adversely affect the transplantation results. In fact, to the contrary, it improved them. If primary myoblast cultures were made fibroblast-free by sub-cloning, it would be envisageable to precondition the donor's myoblasts for a longer time, thereby increasing the number of cells to be transplanted from a relatively small biopsy.

In the capillary bed of the peripheral circulatory system, the enzyme lipoprotein lipase hydrolyzes and removes most of the triglycerides from the chylomicron. The lipoprotein that remains, now rich in cholesterol esters and potentially atherogenic, is called a chylomicron remnant. This postprandial lipoprotein is then removed from the circulation by the liver.

Other products or metabolic agents can be discussed, as such superoxide dismutase, carnitine, creatine, vitamin E, and lipids.

The discovery of mutations to Cu,Zn superoxide dismutase in a subset of familial amyotrophic lateral sclerosis(ALS) cases has raised hopes for understanding the selective vulnerability of motor neurons as well as the pathogenesis of the remaining 98% of ALS cases not related to superoxide dismutase mutations.

Neurofilaments give axons their structural integrity and define axonal diameter. Neurofilaments are composed of three subunits identified as light (NF-L), medium (NF-M) and heavy (NF-H) which assemble in a 6:2:1 ratio to form long macromolecular filaments. Consequently, NF-L is more abundant than the other two subunits in neurons. NF-L is capable of homologous assembly whereas NF-M and NF-H are not competent to assemble in the absence of NF-L. Each neurofilament subunit consists of conserved head and rod domains and a more variable acidic tail domain. The rod domains are principally composed of alpha helixes, which wrap around each other to form a superhelix of parallel coiled coils.

Amyotrophic lateral sclerosis is a fatal neurodegenerative disease characterized by the selective loss of motor neurons and accompanying loss of voluntary muscular function. ALS typically begins as weakness in one limb during middle adult life and progresses via contiguous groups of motor neurons to ultimately result in paralysis and death within 3-5 years. Ninety percent of ALS cases are sporadic with no identifiable genetic or environmental risk factors. A familial inheritance pattern has been observed in the remaining 10% of ALS cases and one-fifth of those result from dominant missense mutations to the antioxidant enzyme copper, zinc superoxide dismutase (Cu,Zn superoxide dismutase). Early histopathological changes in ALS include abnormal accumulations of neurofilaments and other cytoskeletal proteins in the cell soma as well as within proximal axonal swellings. The clinical course and histopathology of sporadic and familial forms of ALS are similar, providing hope that understanding superoxide dismutase-associated ALS was illuminate the pathogenesis of sporadic ALS.

L-carnitine serves two major functions. It is best known for its role in facilitating entry of long-chain fatty acids into mitochondria for utilization in energy-generating processes. Long-chain fatty acids, as coenzyme A esters, are trans-esterified to L-carnitine in a reaction catalyzed by carnitine palmitoyltransferase I of the mitochondrial outer membrane. Long-chain acylcarnitine esters enter into mitochondria via a specific carrier, carnitine-acylcarnitine translocase. On the matrix side of the inner mitochondrial membrane the long-chain fatty acid is transesterified to intramitochondrial coenzyme A, catalyzed by carnitine palmitoyltransferase II. Carnitine may exit the mitochondrion as such or as a short-chain acylcarnitine ester, via the translocase. This function of carnitine is obligatory: long-chain fatty acids cannot enter mitochondria independent of translocation as an ester of carnitine.

L-carnitine also facilitates removal from mitochondria of short-chain and medium-chain fatty acids that accumulate as a result of normal and abnormal metabolism. Short- and medium-chain acids, as acyl-CoA esters arising from β-oxidation and other mitochondrial processes, are trans-esterified to carnitine by the action of carnitine acetyltransferase. The acylcarnitine esters subsequently are transported out of mitochondria by the carnitine acylcarnitine translocase. This pathway provides a means to regenerate intramitochondrial free coenzyme A under conditions where short-chain acyl-CoA esters are produced at a rate faster than they can be utilized.

Pharmacological administration of L-carnitine reduces the mortality and metabolic consequences associated with acute ammonium intoxication in mice. The mechanism associated with this effect may have two components: L-carnitine administration normalizes the redox state of the brain (perhaps by increasing availability of β-hydroxybutyrate to the brain), and it increases the rate of urea synthesis in the liver. At least part of the protective effect of L-carnitine is associated with flux through the carnitine acyltransferases, as inhibition of these enzymes by DL-aminocarnitine, acetyl-DL-aminocarnitine, or palmitoyl-DL-animocarnitine enhances toxicity of acute ammonium administration. Carnitine administration may have significant benefit in patients with disorders of ammonia metabolism, including urea cycle defects, chronic valproic acid therapy, liver failure, organic acidemias, and Reye's syndrome.

It is known that propionyl-L-carnitine protects the ischemic heart from reperfusion injury, perhaps by scavenging free radicals or by preventing their formation by chelating iron necessary for generation of hydroxyl radicals. Long-chain acylcarnitine esters also participate in turnover and repair of erythrocyte membrane phospholipids, independent of ATP hydrolysis. It has been speculated that carnitine and its esters protect cells from oxidative damage, both by inhibiting free-radical propagation and by contributing to repair of oxidized membranes phospholipids. These processes may occur in many cell types, but may be particularly important in cardiac and other red muscle.

In poultry supplemented diet, it is not yet clear if the carnitine and its derivatives have an effect on feed intake, body and abdominal fat weight or on carcass or liver lipid levels.

Vitamin E acts to prevent the production of peroxide lipid as peroxide of an unsaturated fatty acid that is considered to be a material cause of the aging phenomenon. It has also a function of reinforcing blood vessels and activating the bloodstream, provides an anti-stress effect, and is a very important nutrient for human beings and other animals.

On the other hand, in stockbreeding, marine culturing or pet breeding, the problems of aging, reduced disease resistance, stress generation, decreased hatchability, deteriorated egg quality or meat quality, propagation disorder or mastitis, or reduction in the number of somatic cells in milk affect these animals, and a solution of these problems has hitherto been keenly demanded.

In the breeding of useful mammals including livestock animals such as cattle, pigs and horses, and pets such as dogs and cats, and experimental animals such as rats, mice and guinea pigs, reproduction is efficient because these animals are useful for human beings. As the breeding density increases, the acceleration of aging, reduced disease resistance, stress generation, accelerated oxidation of meat foods, deteriorated meat quality such as the blackening of meat foods, and propagation disorder occur more often. Propagation disorder is caused by premature birth, reduction of conception ratio, ovulatory retardation, embryo death, a weakened estrous symptom or reduced production of progesterone.

Poultry such as domestic fowl, quail and turkey under overcrowded breeding conditions suffer from reduced disease resistance, stress generation, deteriorated meat quality and propagation disorder, and additionally, reduced egg quality in the case of egg layers. In order to overcome these problems, various vitamins, including vitamin E and derivatives thereof, and minerals have been conventionally added individually or in combination to the drinking water or feed and then fed to poultry.

Creatine occurs in muscle and nervous tissue (especially in the CNS), and in the form of its secondary metabolite, phosphocreatine, represents an energy reserve for muscle and brain. In the nervous and cardiac muscle tissue creatine appears to have a prophylactic and therapeutic effect in cases of ischemia resulting for instance from infarcts or pre- or perinatal conditions of oxygen deficit.

Creatine is not only an endogenous substance and a valuable food supplement but also has valuable therapeutic properties. It has been known for over a hundred years as a muscular substance and serves as a source of energy for the muscle. It was shown in a series of scientific studies that the intake of creatine can lead to an increase in muscular tissue and muscular performance.

There are also scientific findings that indicate that the pancreas releases more insulin under the influence of creatine. Insulin promotes the uptake of glucose and amino acids by muscle cells and stimulates protein synthesis. Insulin also lowers the rate of protein catabolism.

The prophylactic, therapeutic or dietetic use of creatine in the most varied of application forms (oral, intravenous etc.) necessitates good bioavailability, which in turn means high solubility in water. This requirement is not sufficiently fulfilled in the case of creatine, which, as an amino-acid derivative, is present in the form of an internal salt.

None of the molecule mentioned above, as mature factor or as genetic marker, was considered as involved in the muscular steatosis metabolisms. None of the references disclosed above disclose or suggest the measurement of MSMFs to establish the health status regarding the steatosis, and their use for the treating or alleviating the symptoms of associated disorders. Further, none of the cited references disclose or suggest the administration MSMF alone or in combinations for treating or alleviating the symptoms of the muscular steatosis.

It would be highly desirable to be provided with a new method of modulating factors responsible of modulation of the steatosis status in human and animals. It is to this activity, and its applications in the modulation of steatosis through measurements of MSMF, selecting individuals regarding results of measurements, and administering MSMF to individuals if desired that the present invention be directed.

### SUMMARY OF THE INVENTION

One object of the present invention is to provide a method for prognosis or diagnosis of muscular steatosis based on the level of muscular steatosis-modulating factor (MSMF) in a human or animal, comprising the steps of measuring level of at least one MSMF in a biological sample of a patient, and comparing the patient MSMF level with the MSMF level of a healthy human or animal, wherein a statistically significant difference indicate predisposition or occurrence of steatosis.

According to an object of the present invention, the method is addressed to animals selected from the group consisting of mammal, and avian, and most particularly, the animals selected from the group consisting of porcine, bovine, ovine, caprine, chicken, turkey, horse, goat, canine, and feline.

Identifying differential expression of selected MSMF genes may perform the measurement of MSMF.

The MSMF may be selected from the group consisting of growth hormone, growth factor, cytokine, growth factor receptor, growth hormone receptor, cytokine receptor, and lipid.

The measured MSMF may also be measure of IGF1, IGF2, aFGF, FGF-2, ADRP, IGF1R, PDGFα, TGFβ, TGFα, LPL, EGF, PDGFβ, Leptin, superoxide dismutase, carnitine, creatine kinase, vitamin, or a combination thereof.

MSMF may be measured in a biological sample that may be derived from a sample of blood, serum, plasma, biopsy, fat, salivary, feces, or urine.

Also, measuring level of at least one peptide, a precursor, a metabolite, or a messenger RNA of MSMF performs the method according to the invention.

In accordance with another object, there is provided a method for the treatment of muscular steatosis in a human or animal patient, which comprises regulating MSMF level substantially equivalent to that of healthy patient by administrating an agonist, an antagonist of MSMF, or a combination thereof.

The treatment of steatosis may be performed by administration of an agonist of MSMF that is at least one MSMF.

The agonist may be a recombinant, a precursor, a non-mature, an analog, a purified, or a physiologically active fragment of at least one MSMF.

Also, the agonist of MSMF may be an abzyme.

In another embodiment of the invention, the treatment of steatosis may be performed by administration of antagonists of MSMF that are MSMFs.

Among embodiments of the present invention, the antagonist of MSMF used to treat the steatosis may be an abzyme.

According to the present invention, the antagonist may be selected from the group consisting of antibody, anti-MSMF messenger RNA, MSMF RNA ligand, MSMF-specific antisense primer, anti-MSMF receptor, and mutant MSMF.

Another particular embodiment of the present invention is that agonist, antagonist, or combination thereof may be administered by introducing at least one expression vector into the human or animal.

The expression vector may further be within at least one cell, and the cell is then introduced into a human or an animal to allow the in vivo synthesis of at least one agonist or antagonist of MSMFmay be administered systemically, orally, or intravenously, using an implant, or a slow delivery system.

According to the method of the invention, the muscular steatosis may be caused in an animal for increasing fat content in food, which comprises the step of administrating to the animal a sufficient amount of at least one agonist, antagonist of MSMF, or a combination thereof.

Another object of the invention is that steatosis is caused by administration of agonist of MSMF that is at least one MSMF, or antagonist of MSMF that is at least one inhibitor of MSMF.

The steatosis may be caused by administration of an agonist, or an antagonist selected respectively from the group consisting of recombinant, precursor, non mature, analog, purified, and a physiologically active fragment of at least one MSMF, or an inhibitor of recombinant, precursor, non mature, analog, purified, and a physiologically active fragment of at least one MSMF.

The antagonist according to the method of causing the steatosis may be selected from the group of an antibody, an anti-MSMF messenger RNA, a MSMF RNA ligand, a MSMF-specific antisense primer, an anti-MSMF receptor, a synthetic antisense, a natural antisense, and a mutant MSMF.

The messenger RNA or anti-MSMF messenger RNA may be complementary or corresponding to nucleic acid sequences selected from the group consisting of SEQ ID NO:1 to SEQ ID NO:305, or a fragment thereof.

Agonist of MSMF, antagonist of MSMF, or combination thereof may be administered by introducing at least one expression vector into the human or animal, wherein the expression vector may be within at least one cell, and the cell is then introduced into a host human or animal.

Another object of the method of causing the steatosis, is the administration of an agonist or antagonist systemically, orally, or intravenously, using an implant, or a slow delivery system.

In accordance with the present invention, there is provided a compound of the group of MSMF for treating or inducing muscular steatosis in a human or an animal patient.

The compound may be selected from the group consisting of agonist, antagonist of MSMF, or a combination thereof.

In accordance with the present invention, there is provided a use of a compound of the group of MSMF in the manufacture of a medicament for treating or inducing muscular steatosis.

In accordance with the present invention, there is provided a pharmaceutical composition for use in treating or causing muscular steatosis comprising a therapeutically acceptable and effective amount of a compound of the group of MSMF in association with a pharmaceutically acceptable carrier.

For the purpose of the present invention the following terms are defined below.

The term “growth factor” as used herein refers to any receptor ligand that causes a cell growth and/or cell proliferation effect. Examples of growth factors are well known in the art. Fibroblast growth factor (FGF) is one example of a growth factor.

The term “recombinant product” as used herein refers to the product produced from a DNA sequence that comprises at least a portion of the MSMF. This product can be a peptide, a polypeptide, a protein, an enzyme, an antibody, an antibody fragment, a polypeptide that binds to a regulatory element (a term described hereafter), a structural protein, an RNA molecule, and/or a abzyme, for example. These products are well defined in the art.

By “expression vector” is meant any nucleic acid molecule or virus containing regulatory elements or reporter genes for the purpose of expression of a given gene in prokaryotic or eukaryotic cells or organisms. Such vectors can be introduced into a cell by means of molecular biological techniques. After introduction into the cell, this nucleic acid can exist extrachromosomally or become integrated into the host genome.

The term “abzyme” as used herein means antibody directed enzyme prodrug. Abzymes are defined as antibodies directed against appropriate transition state analogues that can catalyse a variety of chemical transformations and metabolic reactions. Furthermore, murine antibodies can be “humanized” using existing technologies to reduce their immunogenicity in patients. Thus a humanized catalytic antibody (abzyme) could be prepared which replaces an enzyme and thus leads to a treatment system that combines both specificity and low immunogenicity.

Unless otherwise defined, all technical and scientific terms used herein have the same meaning as commonly understood by one of ordinary skill in the art to which this invention belongs. Although methods and materials described herein can be used to practice the present invention, other similar or equivalent methods and material known to one skilled in the art can also be used. All publications, patent applications, patents, and other references mentioned herein are incorporated by reference in their entirety. In case of conflict, the present specification, including definitions, was control. The materials, methods, and examples described herein are illustrative only and not intended to be limiting.

### BRIEF DESCRIPTION OF THE DRAWING

FIG. 1 illustrates level expression (RT-PCR) of studied genes for muscular fat as steatosis markers in healthy pigs and pigs having high degree of steatosis.

### DETAILED DESCRIPTION OF THE INVENTION

In accordance with the present invention, there is provided a new method of modulating levels of muscular steatosis-modulating factors (MSMF), measuring body levels of human and animal with naturally occurring or administered MSMF. As shown in examples provided below, measurement of steatosis based on measured levels of MSMF could be made by comparison to levels measured in a steatosis-free control group or background level measured in a particular subject. The measurement can be confirm by correlation of the assay results with other aforementioned methods of disease known to those skilled in the arts, such as photonic microscopy. Among MSMFs of the present invention, there is considered acidic and basic fibroblast growth factor (aFGF, FGF-2), transforming growth factor -beta and -alpha (TGF-β and TGF-α), adipocyte differentiating related protein (ADRP), epidermal growth factor (EGF), insulin like growth factor 1 and 2 (IGF-1 and IGF-2), IGF-1 receptor and IGF-2 receptor, platelet derived growth factor -alpha and -beta (PDGF-α and PDGF-β), leptin, and lipoprotein lipase (LPL). Lipids that can be considered in establishing the steatosis status for monitoring MSMF of an individual are myristic acid (C14:0), myristoleic acid (C14:1), pentadecanoic acid (C15:0), pentadecenoic acid (C15:1), palmitic acid (C16:0), palmitoleic acid (C16:1), margaric acid (C17:0), margaroleic acid (C17:1), stearic acid (C18:0), oleic acid (C18:1), linoleic acid (C18:2), linoleinic acid (C18:3), arachidic acid (C20:0), eicosenoic acid (C20:1), eicosadienoic acid (C20:2), eicosatrienoic acid (C20:3), arachidonic acid (C20:4), beneic acid (C22:0), erucic acid (C22:1), docosandienoic acid (C22:2), docosahexaenoic acid (C22:6), and lignoceric acid (C24:0).

In another embodiment of the invention, there is provided a method of detecting and quantifying MSMF in biological samples using an antibody specific for MSMF and, where appropriate, a detectable-labeled antigen (MSMF). The invention is to provide methods for diagnosis of diseases that are correlated to the loss and/or synthesis of muscular tissue as indicated by levels of MSMF or lipids detected in a biological sample. A method of identifying differential expression of selected genes is used to diagnosing the muscular steatosis in human and animals.

In another embodiment of the present invention, there is provided measure levels of FGF-2, IGF1R and LPL alone or in combinations as genetic markers in determining sings of muscular steatosis in a human or an animal.

In another embodiment of the present invention, there is provided a method for determining the steatosis status by using reverse transcription and polymerase chain reaction to amplify small amounts of MSMF mRNA. DNA-DNA hybridization can then be used to confirm the specificity of the amplified product as being MSMF.

This technique provides a method for measuring the quantities of MSMF. The ability to demonstrate the quantities of MSMF by RT-PCR and then confirm the specificity of the amplification by DNA hybridization has significant implications in clarifying MSMF role in muscular steatosis. In practice it is rendered possible a direct testing of biological samples for the presence of MSMF that may be conducted.

The invention further provides screening methods to identify concentration of molecules that can be involved in modulating steatosis. In one aspect, such screening methods comprise competitive binding assays wherein the ability of a putative modulating molecule to bind to MSMF is measured in the presence of a suitably labeled C-terminal peptide.

In one embodiment of the invention, MSMF are measured to selected animals having specific characteristics regarding targeted MSMF. Those animals selected to be exempted of any sing of steatosis may be considered as genetically qualified for establishing lineages. For example, farm production of porcine, bovine, chicken, turkey, ovine and caprine should profit of genetically selected founders in the establishment of healthy herds through the present invention.

In another embodiment, the invention is directed to the selection of stably genetically selected individuals having naturally different status of muscular steatosis, to serve as founder animals for the establishment of specific herds having these properties. It is well recognized that lipids and ratios of muscular lipids can influence the texture and taste of the meat. In some cases, higher level of muscular steatosis may be suitable to have animal with more fatty muscles.

Alternatively, agonists of positively inducing MSMF, or MSMF itself can be administered to an animal to induce steatosis for the same aim mentioned above. Agonists of MSMF, for example, can be a MSMF itself or combinations of MSMF, or abzymes that mimic binding sites of MSMF to their respective cell receptors, or that mimic enzymatic activity of the MSMF. Antagonists of MSMF, can be administered to reestablish a healthy state of an individual affected by the muscular steatosis.

Yet additional embodiments of the invention comprise the use of MSMF and lipid compositions of the invention as screening markers for molecules which modulate or are involved in the establishment of muscular steatosis. Such embodiments include, but are not limited to, assays which measure the ability of a putative MSMF to compete with other peptides and proteins (including, but not limited to, other peptide sequences of the MSMF itself), which are identified to act specifically to the receptor compositions of the invention, in order to modulate the steatotic state of an individual.

The immunoassay procedure used is preferably quantitative so that levels of MSMF in a patient with disease may be distinguished from normal levels which may be present in healthy individual and/or background levels measured in the patient. Competitive and sandwich assays on a solid phase using detectable labels (direct or indirect) are, therefore, preferred. The label provided a detectable signal indicative of binding of antibody to the MSMF antigen. The antibody or antigen may be labeled with any label known in the art to provide a detectable signal, including radioisotopes, enzymes, fluorescent molecules, chemiluminescent molecules, bioluminescent molecules and colloidal gold. Of the known assay procedures, radioimmunoassay (RIA) is most preferred for its sensitivity. A radioisotope had, therefore, is the preferred label.

It has been appreciated by those skilled in the art that, although not necessarily as sensitive as an RIA, assay procedures using labels other than radioisotopes have certain advantages and may, therefore, be employed as alternatives to the preferred RIA format. For example, an enzyme-linked immunosorbent assay (ELISA) may be readily automated using an ELISA micrometer plate reader and reagents who are readily available in many research and clinical laboratories. Fluorescent, chemiluminescent and bioluminescent labels have the advantage of being visually detectable, though they are not as useful as radioisotopes to quantify the amount of antigen bound by antibody in the assay.

Molecules identified by means of the screening assays of the invention has been candidates as useful therapeutic products for the in vivo, ex vivo or in vitro treatment of target tissues alone or in combination with suitable carriers and excipients. Such compositions and their use comprise additional embodiments of the invention.

In yet another embodiment of the invention, there is provided expression vectors containing genetic sequences, hosts transformed with such expression vectors, and methods for producing the recombinant MSMF compositions of the invention.

The present invention is further directed to methods for inducing or suppressing apoptosis in the cells and/or tissues of individuals suffering from disorders characterized by inappropriate cell proliferation or survival, or by inappropriate cell death, respectively. Disorders characterized by inappropriate cell proliferation and/or survival include, for example, inflammatory conditions, cancer, including lymphomas, genotypic tumors, etc. Disorders characterized by inappropriate cell death include, for example, autoimmune diseases, acquired immunodeficiency disease (AIDS), cell death due to radiation therapy or chemotherapy, acute hypoxic injury, etc.

In another embodiment of the present invention, there is provided a method for identification of the hormones and other factors, the steatosis-modulating factors, controlling the balance between muscular and adipocyte proliferation and differentiation, that is very important for modulating normal adipose and muscular tissue development and for designing approaches for screening individuals having normal and abnormal states of adipose tissue development, such as obesity for example.

In yet another embodiment of the present invention, there is provided a method of treating an individual with MSMF in an individual that need such treatment, comprising the step of administering to the individual a pharmacologically effective dose of one MSMF aforementioned or combinations thereof.

The present invention was more readily understood by referring to the following examples, which are given to illustrate the invention rather than to limit its scope.

### EXAMPLE I

### Muscle-Fat Imbalancement in Growing Pigs

In swine, specific ham muscles such as semi-tendineous biceps femoris and semi-membranous are sometimes abnormally infiltrated with fat, leading to a severe muscle degeneration. We suspected different genetic factors to be implicated in the development of this muscular-fat imbalance.

A total of 113 among 676 pigs were selected in a local farm. Healthy and steatotic animals were directly selected at the farm by using ALOKA apparatus performing bi-directional ultrasonic reading. After slaughter, 80 pigs were retained following a visual quotation of the left semi-tendineous muscle and according to a design with muscular fat infiltration (0 or severe).

Methods of Analysis

Vitamin E: The concentrations of plasmatic and hepatic Vitamin E were determined through an home made adapted method described by Bieri et al (Bieri, J. G. et al., Am. J. Clin. Nutri. (1979) vol. 32; 2143-2149) on HPLC (High Pressure Liquid Chromatography). Results are presented in Table 1.

L-carnitine: Using a modified approaches (radio-isotopical) developed by McGarry and Foster ((1976) J. Lipid Res. 17:277-281), concentrations of L-carnitine contained in semi-tendineous muscles and plasma were determined. Results are shown in Table 1.

Creatine kinase: The analysis of quantities of creatine kinase in plasma has been performed with a commercial (Sigma Diagnostics #C2527, St-Louis, Mo.) enzymatic kit allowing to measure variations of NADH at optical density of 340 nm, that is a direct indication of the creatine kinase activity. Results are presented in Table 1.

Selenium: Quantities of selenium in blood was directly measured by the assessment of the activity of glutathion peroxidase. The level of activity is determined by measuring oxidative rates of NADPH by spectrophotometry at 340 nm of optical density. Results are presented in Table 1.

Superoxide dismutase: The activity of superoxide-dismutase in muscles was performed with a commercial kit (Calbiochem, #574600, San Diego, Calif.) allowing to measure variation in levels of oxidation of a chromophore agent by optical density at 525 nm. Results are shown in Table 1.

TABLE 1
SUMMARY OF STATISTICS OF BLOOD AND TISSUE ANALYSIS

| | SEX | | STEATOSIS | | | P | | |
|---|---|---|---|---|---|---|---|---|
| Variables | M | F | Affected | Normal | SEM | Sex | Steatosis | S/ST |
| CARNITINE | 22.01 | 21.32 | 21.52 | 21.81 | 0.644 | 0.451 | 0.753 | 0.574 |
| MUSCLE | | | | | | | | |
| (nmoles/mg | | | | | | | | |
| protein) | | | | | | | | |
| CARNITINE | 4.67 | 4.42 | 4.88 | 4.21 | 0.213 | 0.408 | 0.029 | 0.656 |
| PLASMA (umoles/ | | | | | | | | |
| liter) | | | | | | | | |
| CREATINE KINASE | 382.0 | 418.4 | 360.9 | 439.6 | 49.625 | 0.613 | 0.276 | 0.536 |
| PLASMA | | | | | | | | |
| (Unit/liter) | | | | | | | | |
| SELENIUM | 17.002 | 17.724 | 17.527 | 17.199 | 0.769 | 0.508 | 0.764 | 0.558 |
| PLASMA | | | | | | | | |
| (nmoles/mg | | | | | | | | |
| protein) | | | | | | | | |
| SELENIUM | 231.323 | 222.356 | 228.020 | 225.658 | 9.019 | 0.484 | 0.854 | 0.456 |
| BLOOD | | | | | | | | |
| (umoles/min./gram | | | | | | | | |
| Hb) | | | | | | | | |
| SUPEROXIDE | 18.66 | 19.42 | 18.41 | 19.68 | 0.284 | 0.062 | 0.002 | 0.894 |
| DISMUTASE | | | | | | | | |
| MUSCLE | | | | | | | | |
| (Unit/mg | | | | | | | | |
| protein) | | | | | | | | |
| VITAMIN E LIVER | 20.886 | 20.243 | 22.691 | 18.438 | 0.859 | 0.600 | 0.001 | 0.279 |
| (ug/gram liver) | | | | | | | | |
| VITAMINE E | 1.188 | 1.371 | 1.268 | 1.291 | 0.064 | 0.050 | 0.803 | 0.957 |
| PLASMA | | | | | | | | |
| (ug/ml) | | | | | | | | |

Legend: M, males; F, females; S/ST, Sex*Steatosis; SEM, standard deviation/□n; (n = 40) P, probability; significant when < 0.05 (shaded)

Patterns of muscular and sub-cutaneous fatty acids were determined on gas phase chromatography. Results are shown in Tables 2, 3 and 4.

TABLE 2
BACKFAT AND MUSCLE TISSUE FATTY ACIDS (%) IN NORMAL
AND AFFECTED PIGS

| Tissue | SEX | | STEATOSIS | | | P | | |
|---|---|---|---|---|---|---|---|---|
| Fat | M | F | Affected | Normal | SEM | Sex | Steatosis | S/ST |
| FAT | 48.66 | | 49.62 | 49.21 | 0.485 | 0.040 | 0.560 | 0.018 |
| MONO | | | | | | | | |
| FAT | 17.14 | | 16.34 | 17.57 | 0.492 | 0.605 | 0.092 | 0.971 |
| POLY | | | | | | | | |
| FAT | 34.20 | | 34.04 | 33.22 | 0.526 | 0.141 | 0.283 | 0.029 |
| SATURED | | | | | | | | |
| MUSCLE | 47.58 | | 48.51 | 46.29 | 1.136 | 0.819 | 0.182 | 0.034 |
| MONO | | | | | | | | |
| MUSCLE | 14.39 | | 11.92 | 18.33 | 0.727 | 0.170 | 0.001 | 0.105 |
| POLY | | | | | | | | |
| MUSCLE | 38.01 | | 39.56 | 35.35 | 0.956 | 0.422 | 0.005 | 0.182 |
| SATURED | | | | | | | | |

Legend: M, males; F, females; S/ST, Sex*Steatosis; SEM, standard deviation/√n; (n = 12) P, probability; significant when < 0.05 (shaded).

TABLE 3
BACKFAT FATTY ACID COMPOSITION (%) IN NORMAL AND
AFFECTED PIGS

| FATTY | | SEX | | STEATOSIS | | | P | | |
|---|---|---|---|---|---|---|---|---|---|
| ACIDS | TISSUE | M | F | Affected | Normal | SEM | Sex | Steatosis | S/ST |
| C14:0 | FAT | 1.54 | 1.34 | 1.34 | 1.54 | 0.061 | 0.025 | 0.031 | 0.001 |
| C14:1 | FAT | 0.08 | 0.06 | 0.05 | 0.10 | 0.015 | 0.341 | 0.041 | 0.296 |
| C15:0 | FAT | 0.15 | 0.11 | 0.10 | 0.16 | 0.022 | 0.172 | 0.060 | 0.180 |
| C15:1 | FAT | 0.08 | 0.07 | 0.05 | 0.09 | 0.010 | 0.368 | 0.024 | 0.252 |
| C16:0 | FAT | 22.09 | 20.80 | 21.28 | 21.60 | 0.448 | 0.056 | 0.618 | 0.006 |
| C16:1 | FAT | 2.89 | 2.77 | 2.57 | 3.09 | 0.087 | 0.324 | 0.001 | 0.007 |
| C17:0 | FAT | 0.42 | 0.37 | 0.38 | 0.41 | 0.022 | 0.068 | 0.266 | 0.108 |
| C17:1 | FAT | 0.36 | 0.36 | 0.34 | 0.39 | 0.020 | 0.863 | 0.090 | 0.807 |
| C18:0 | FAT | 9.37 | 9.88 | 10.41 | 8.83 | 0.194 | 0.079 | 0.001 | 0.020 |
| C18:1 | FAT | 42.87 | 44.36 | 43.46 | 43.77 | 0.510 | 0.053 | 0.669 | 0.136 |
| C18:2 | FAT | 14.47 | 13.97 | 13.72 | 14.73 | 0.444 | 0.434 | 0.124 | 0.985 |
| C18:3 | FAT | 1.29 | 1.29 | 1.21 | 1.36 | 0.051 | 0.972 | 0.045 | 0.567 |
| C20:0 | FAT | 0.22 | 0.24 | 0.24 | 0.22 | 0.011 | 0.239 | 0.295 | 0.070 |
| C20:1 | FAT | 2.23 | 2.39 | 3.05 | 1.57 | 0.585 | 0.848 | 0.089 | 0.231 |
| C20:2 | FAT | 0.61 | 0.70 | 0.67 | 0.65 | 0.028 | 0.039 | 0.644 | 0.053 |
| C20:3 | FAT | 0.17 | 0.16 | 0.15 | 0.18 | 0.012 | 0.650 | 0.128 | 0.781 |
| C20:4 | FAT | 0.26 | 0.32 | 0.29 | 0.29 | 0.022 | 0.058 | 0.792 | 0.096 |
| C22:0 | FAT | 0.15 | 0.16 | 0.13 | 0.17 | 0.039 | 0.830 | 0.453 | 0.306 |
| C22:1 | FAT | 0.14 | 0.17 | 0.10 | 0.21 | 0.028 | 0.461 | 0.015 | 0.506 |
| C22:2 | FAT | 0.16 | 0.17 | 0.14 | 0.20 | 0.020 | 0.849 | 0.044 | 0.450 |
| C22:6 | FAT | 0.17 | 0.16 | 0.17 | 0.17 | 0.026 | 0.795 | 0.925 | 0.026 |
| C24:0 | FAT | 0.24 | 0.17 | 0.15 | 0.27 | 0.033 | 0.097 | 0.023 | 0.494 |

Legend: M, males; F, females; S/ST, Sex*Steatosis; SEM, standard deviation/√n; (n = 12) P, probability; significant when < 0.05 (shaded).

TABLE 4
MUSCLE FATTY ACID COMPOSITION (%) IN NORMAL AND
AFFECTED PIGS

| FATTY | | SEX | | STEATOSIS | | | P | | |
|---|---|---|---|---|---|---|---|---|---|
| ACID | TISSUE | M | F | Affected | Normal | SEM | Sex | Steatosis | S/ST |
| C14:0 | MUSCLE | 1.76 | 1.36 | 1.84 | 1.28 | 0.118 | 0.028 | 0.003 | 0.216 |
| C14:1 | MUSCLE | 0.15 | 0.13 | 0.09 | 0.18 | 0.026 | 0.583 | 0.025 | 0.101 |
| C15:0 | MUSCLE | 0.23 | 0.13 | 0.17 | 0.19 | 0.052 | 0.158 | 0.731 | 0.186 |
| C15:1 | MUSCLE | 0.53 | 0.63 | 0.27 | 0.89 | 0.074 | 0.390 | 0.001 | 0.026 |
| C16:0 | MUSCLE | 24.17 | 22.69 | 25.42 | 21.44 | 0.764 | 0.185 | 0.001 | 0.197 |
| C16:1 | MUSCLE | 3.45 | 3.08 | 3.43 | 3.11 | 0.128 | 0.053 | 0.095 | 0.736 |
| C17:0 | MUSCLE | 0.33 | 0.31 | 0.28 | 0.36 | 0.021 | 0.576 | 0.014 | 0.081 |
| C17:1 | MUSCLE | 0.28 | 0.28 | 0.27 | 0.29 | 0.015 | 0.839 | 0.266 | 0.195 |
| C18:0 | MUSCLE | 10.60 | 11.47 | 11.26 | 10.81 | 0.330 | 0.077 | 0.348 | 0.634 |
| C18:1 | MUSCLE | 41.45 | 41.44 | 42.87 | 40.02 | 1.217 | 0.995 | 0.114 | 0.065 |
| C18:2 | MUSCLE | 10.77 | 11.99 | 9.31 | 13.45 | 0.525 | 0.116 | 0.001 | 0.340 |
| C18:3 | MUSCLE | 0.88 | 0.90 | 0.82 | 0.96 | 0.046 | 0.775 | 0.044 | 0.675 |
| C20:0 | MUSCLE | 0.29 | 0.31 | 0.26 | 0.34 | 0.042 | 0.693 | 0.206 | 0.357 |
| C20:1 | MUSCLE | 1.44 | 1.51 | 1.42 | 1.53 | 0.387 | 0.909 | 0.845 | 0.171 |
| C20:2 | MUSCLE | 0.54 | 0.55 | 0.51 | 0.59 | 0.037 | 0.915 | 0.151 | 0.044 |
| C20:3 | MUSCLE | 0.33 | 0.32 | 0.22 | 0.43 | 0.046 | 0.930 | 0.004 | 0.050 |
| C20:4 | MUSCLE | 1.24 | 1.62 | 0.60 | 2.26 | 0.203 | 0.201 | 0.001 | 0.046 |
| C22:0 | MUSCLE | 0.21 | 0.22 | 0.12 | 0.31 | 0.034 | 0.931 | 0.001 | 0.045 |
| C22:1 | MUSCLE | 0.27 | 0.15 | 0.17 | 0.26 | 0.045 | 0.078 | 0.161 | 0.290 |
| C22:2 | MUSCLE | 0.32 | 0.28 | 0.19 | 0.41 | 0.043 | 0.445 | 0.002 | 0.360 |
| C22:6 | MUSCLE | 0.32 | 0.21 | 0.28 | 0.25 | 0.049 | 0.138 | 0.621 | 0.066 |
| C24:0 | MUSCLE | 0.41 | 0.41 | 0.21 | 0.61 | 0.074 | 0.953 | 0.001 | 0.236 |

Legend: MUSCLE, affected part of the muscle; M, males; F, females; S/ST, Sex*Steatosis; SEM, standard deviation/√n; (n = 12) P, probability; significant when < 0.05 (shaded).

PCR Amplification of Messenger RNA (RT-PCR):

Separation of intramuscular fat and muscle fibers: In order to amplify the transcripts that correspond only to intramuscular fat or to muscle fibers, pieces of semi-tendineous were taken from the freezer and immediately placed under a binocular. Separation of intramuscular fat from muscle fibers was performed manually using a thin needle. Samples of intramuscular fat and muscle fibers were immediately transferred to tubes filled with 2 ml Trizol™ reagent (Gibco-BRL, Bethesda, Md.). These tubes were kept at −80° C. until needed.

RNA extraction: RNA was extracted in Trizol™ reagent according to the manufacturer's instructions. The extracted RNA was dissolved in water and quantified spectrophotometrically at 260 nm. RNA aliquots were electrophoresed in a 1% agarose gel to verify their integrity.

Quantitative RT-PCR: For all samples, 5 μg of RNA was treated with 3 units of Dnase I (Amplification grade #8068-015, Gibco-BRL, Bethesda, Md.) to remove contaminating genomic DNA. First strand cDNA was synthesized from 5 μg of total RNA from either intramuscular fat or muscle fibers, using a SuperScript™ II preamplification system for first strand cDNA synthesis (Gibco BRL, Burlington, ON) and 500 ng of oligo (dT) 12-18 primer in a total reaction volume of 50 μl. An aliquot of 2 μl of the reverse transcriptase product was subjected to PCR amplification.

The following RT-PCR were performed for intramuscular fat: ADRP, EGF, IGF1, IGF2 IGF1R, IGF2R, PDGFα, PDGFβ, TGFβ, aFGF, FGF-2, TGFα, leptin, LPL and MEF2 as a control. In muscle fibers the following RT-PCR were performed: EGF, IGF1, IGF2 IGF1R, IGF2R, PDGFα, PDGFβ, TGFβ, aFGF, FGF-2, TGFα, LPL and leptin as a control.

For each gene, a 100 μl PCR reaction contained either 15 pmol or 30 pmol of upstream and downstream primers (see Table 5), 200 μM dNTPs, 1.5 mM MgCl₂ and 2.5 units of Taq™ polymerase in 1×Taq” polymerase buffer (Amersham Pharmacia Biotech, Baie d'Urfée, QC). Each gene's PCR profile was performed using a Programmable Thermal Controller PTC-100™ (MJ Research Inc., Watertown, Mass.). The PCR amplifications consisted of an initial denaturation step at 94° C. for 2 min, followed by variable cycle numbers of denaturation at 94° C. for 1 min (see Table 5), annealing at different temperature for 1 min (see Table 5), extension at 72° C. for 1 min and a final extension at 72° C. for 5 min. Pig glyceraldehyde-3-phosphate dehydrogenase (GAPDH) was also used as an internal control of amplification. For GAPDH PCR amplification, the 100 μl PCR reaction contained 30 pmol of upstream and 30 pmol of downstream primers (Table 5), 200 μl dNTPs, 1.5 mM MgCl₂ and 2.5 units of Taq™ polymerase in 1×polymerase buffer. The GAPDH PCR profile consisted of an initial denaturation step at 94° C. for 2 min, followed by 20 cycles of denaturation at 94° C. for 1 min, annealing at 68° C. for 1 min, extension at 72° C. for 1 min and a final extension at 72° C. for 5 min.

TABLE 5

PCR Conditions

| | | | | | | | | |
|---|---|---|---|---|---|---|---|---|
| | | | | | | | Concen- | Hybrida- |
| | | | | | | | tration | tion |
| | Primer | Primer Sequence | Position | size | Genebank | cycle | oligo | Tempera- |
| Sondes | Site | (5′-3′) | (pb) | fragments | no | numbers | (pmoles) | ture |

| | | | | | | | | |
|---|---|---|---|---|---|---|---|---|
| IGF 1 | Forward | 5′-GCA CAT CAC | 15-37 | 338 | PIGGFIIA | 25 | 30 | 70 C. |
| | | ATC CTC TTC GCA | | | | | | |
| | | TC-3′ | | | | | | |
| | Reverse | 5′-TGT ACT TCC | 331-353 | | | | | |
| | | TTC TGA GCC TTG | | | | | | |
| | | GG-3′ | | | | | | |
| IGF 2 | Forward | 5′-GGT GGA CAC | 481-502 | 354 | SSIGF2 | 24 | 15 | 70 C. |
| | | CCT CCA GTT TGT | | | | | | |
| | | C-3′ | | | | | | |
| | Reverse | 5′-GTG ACG CTT | 815-835 | | | | | |
| | | GGC CTC TCT GAC-3′ | | | | | | |
| IGF1R | Forward | 5′-CGC ATG TGC | 19-40 | 307 | SSU15445 | 25 | 30 | 70 C. |
| | | TGG GAG TAC AAC | | | | | | |
| | | C-3′ | | | | | | |
| | Reverse | 5′-TGC GCG TAA | 306-326 | | | | | |
| | | GGC TGT CTC TCG-3′ | | | | | | |
| IGF2R | Forward | 5′-GGC CAA GTC | 1-22 | 381 | S5U58650 | 25 | 30 | 70 C. |
| | | CAA CTG CCG CTA | | | | | | |
| | | C-3′ | | | | | | |
| | Reverse | 5′-ACT CAT CCG | 363-382 | | | | | |
| | | CTG GAA GCC CG-3′ | | | | | | |
| aFGF | Forward | 5′-TGG CTG AAG | 17-39 | 414 | SSAFGFRNA | 25 | 30 | 70 C. |
| | | GCG AAA TCA CAA | | | | | | |
| | | CC-3′ | | | | | | |
| | Reverse | 5′-TGA GTC CGA | 411-431 | | | | | |
| | | GGA CCG CGT TTG-3′ | | | | | | |
| bFGF | Forward | 5′-ACG GAG GCT | 138-157 | 283 | SSBFGF | 24 | 15 | 70 C. |
| | | TCT TCC TGC GC-3′ | | | | | | |
| | Reverse | 5′-CGT TCG TTT | 399-421 | | | | | |
| | | CAG TGC CAC GTA | | | | | | |
| | | CC-3′ | | | | | | |
| EGF | Forward | 5′-ATC GGTACC | 2607-2630 | 899 | HSEGFRER | 25 | 15 | 65 C. |
| | | GCA TGC TGA AGC | | | | | | |
| | | CCT CAT CAC TGG-3′ | | | | | | |
| | Reverse | 5′-ATC TCTAGA | 3482-3505 | | | | | |
| | | GCG CAG CTC CCA | | | | | | |
| | | CCA TTT CAA GTC-3′ | | | | | | |
| TGFa | Forward | 5′-CTT GTT GGC | 54-74 | 411 | SSTGFA | 27 | 15 | 70 C |
| | | CGT GTG CCA GGC-3′ | | | | | | |
| | Reverse | 5′-AGC GGT CCT | 443-465 | | | | | |
| | | TCC CTT CAG GAG | | | | | | |
| | | GG-3′ | | | | | | |
| TGFb | Forward | 5′-AAG CGG AAG | 570-588 | 995 | GGTGFB1 | 25 | 30 | 70 C. |
| | | CGC ATC GAG G-3′ | | | | | | |
| | Reverse | 5′-GCG GCC CAC | 1547-1565 | | | | | |
| | | GTA GTA CAC G-3′ | | | | | | |
| PDGFa | Forward | 5′-CCC GCG AGG | 464-482 | 408 | HSPDGFAR | 24 | 15 | 70 C. |
| | | TGA TCG AGA G-3′ | | | | | | |
| | Reverse | 5′-GGC TTC TTC | 850-872 | | | | | |
| | | CTG ACG TAT TCC | | | | | | |
| | | AC-3′ | | | | | | |
| PDGFb | Forward | 5′-CTC TGC TGC | 1013-1036 483 | HSPDGFB | 24 | 15 | 70 C. | |
| | | TAC CTG CGT CTG | | | | | | |
| | | GTC-3′ | | | | | | |
| | Reverse | 5′-GCG TCA CCG | 1474-1496 | | | | | |
| | | TGG CCT TCT TAA | | | | | | |
| | | AG-3′ | | | | | | |
| ADRP | Forward | 5′-ATC AAGCTT | 27-50 | 1173 | D | 27 | 15 | 70 C. |
| | | AAC AGA GCG TGG | | | MMADRPCO | | | |
| | | TGA TGA GAG TGG | | | | | | |
| | | C-3′ | | | | | | |
| | Reverse | 5′-ATC TCTAGA | 1181-1200 | | | | | |
| | | CCT ACC AGC CAG | | | | | | |
| | | TTG AGA GGC G-3′ | | | | | | |
| LEPTIN (ob) | Forward | 5′-GTC GAT TCC | 74-96 | 409 | AF026976 | 24 | 15 | 70 C. |
| | | TGT GGC TTT GGC | | | | | | |
| | | CC-3′ | | | | | | |
| | Reverse | 5′-CTC CGT GGA | 459-483 | | | | | |
| | | GTA GAG GGA GGC | | | | | | |
| | | TTC C-3′ | | | | | | |
| LEPTIN (ob) | Forward | 5′-GTC GAT TCC | 74-96 | 409 | AF026976 | 35 | 15 | 70 C. |
| | | TGT GGC TTT GGC | | | | | | |
| | | CC-3′ | | | | | | |
| (control) | Reverse | 5′-CTC CGT GGA | 459-483 | | | | | |
| | | GTA GAG GGA GGC | | | | | | |
| | | TTC C-3′ | | | | | | |
| MEF2 | Forward | 5′-GCA TGA TGC | 1262-1284 | 535 | HSMEF2 | 35 | 30 | 70 C. |
| | | CTC CAC TAT CGG | | | | | | |
| | | AG-3′ | | | | | | |
| (control) | Reverse | 5′-AGA GCT GCT | 1774-1797 | | | | | |
| | | GAG ACT GTC CAC | | | | | | |
| | | AGG-3′ | | | | | | |
| GAPDH | Forward | 5′-CTG GCA AAG | 28-50 | 571 | SSU48832 | 20 | 30 | 68 C. |
| | | TGG ACA TTG TCG | | | | | | |
| | | CC-3′ | | | | | | |
| | Reverse | 5′-CTT GGC AGC | 579-599 | | | | | |
| | | GCC GGT AGA AGC-3′ | | | | | | |
| Lipoproteine | Forward | 5′-GAG GGA ACC | 475-494 | 709 | SSLPLRNA | 24 | 30 | 65 C. |
| | | GGA TTC CAA CG-3′ | | | | | | |
| Lipase (LPL) | Reverse | 5′-AGG GCA TCT | 1165-1184 | | | | | |
| | | GAG CAC GAG TC-3′ | | | | | | |

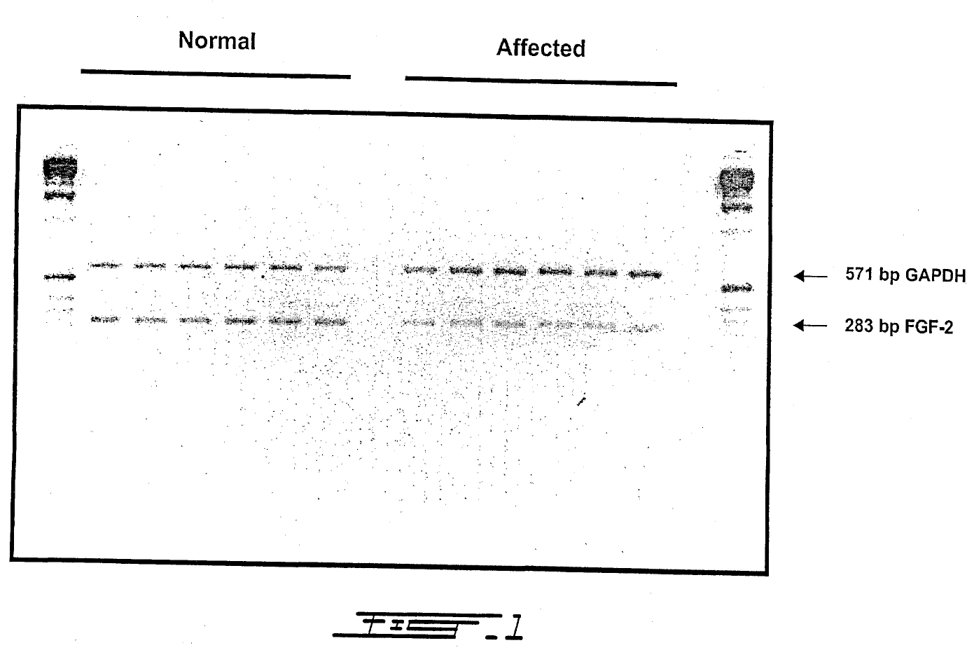

The amplified PCR fragments were electrophoresed on a 2.5% agarose gel and stained with ethidium bromide. Pictures of the resulting gels were taken on Polaroid film # 55. Films were then scanned using a densitometer (BIO-RAD™ Imaging Densitometer Model GS-670 Bio-Rad Laboratories Led., Mississauga, ON). The relative optical density of the transcripts is expressed in arbitrary optical units. A ratio of the optical density of each transcript, standardized using the GAPDH transcript, was calculated before statistical analyses were performed to correct for possible differences in gel loading. The results are shown in Tables 6, 7, 8 and in FIG. 1 representing the RT-PCR analysis of FGF-2 gene expression in muscle fibers of healthy (normal) and steatotic (affected) pigs, and where is amplified mRNA specific to the genes GAPDH (fragment of 571 bp) as control, and FGF-2 (fragment of 282 bp) as differential MSMF marker.

TABLE 6
EXPRESSION LEVELS (RT-PCR) OF DIFFERENT CANDIDATE
GENES IN INTRAMUSCULAR FAT OF NORMAL AND
AFFECTED PIGS

| | | | | | | P | | |
|---|---|---|---|---|---|---|---|---|
| | SEX | | STEATOSIS | | | | Stea- | |
| GENE | M | F | Affected | Normal | SEM | Sex | tosis | S/ST |
| ADRP | 44.86 | 58.75 | 47.09 | 56.52 | 6.160 | 0.127 | 0.292 | 0.340 |
| EGF | 67.24 | 56.87 | 47.61 | 76.50 | 6.107 | 0.244 | 0.003 | 0.059 |
| IGF1 | 55.10 | 69.78 | 65.19 | 59.69 | 4.686 | 0.039 | 0.417 | 0.518 |
| IGF1R | 59.24 | 64.79 | 71.78 | 52.24 | 3.354 | 0.256 | 0.001 | 0.830 |
| IGF2 | 45.70 | 51.78 | 34.09 | 63.38 | 6.826 | 0.536 | 0.007 | 0.931 |
| IGF2R | 76.01 | 65.97 | 75.38 | 66.60 | 6.644 | 0.298 | 0.361 | 0.258 |
| PDGFα | 76.44 | 72.06 | 74.01 | 74.48 | 4.689 | 0.516 | 0.944 | 0.697 |
| PDGFβ | 65.28 | 53.92 | 56.82 | 62.38 | 7.238 | 0.280 | 0.593 | 0.163 |
| TGFβ | 58.62 | 68.10 | 55.30 | 71.42 | 7.473 | 0.381 | 0.143 | 0.945 |
| aFGF | 69.74 | 59.74 | 56.22 | 73.26 | 5.128 | 0.183 | 0.029 | 0.198 |
| FGF-2 | 37.79 | 51.46 | 53.38 | 35.87 | 3.198 | 0.007 | 0.001 | 0.001 |
| TGFα | 45.07 | 63.72 | 66.52 | 42.27 | 3.350 | 0.001 | 0.001 | 0.010 |
| Leptin | 53.50 | 64.15 | 68.81 | 48.83 | 3.231 | 0.030 | 0.001 | 0.040 |
| LPL | 76.23 | 60.19 | 65.92 | 70.49 | 5.389 | 0.048 | 0.556 | 0.281 |
| Legend: M, males; F, females; S/ST, Sex*Steatosis; SEM, standard deviation/√n; P, probability; significant when < 0.05 (shaded). Values in this table correspond to relative optical density that were adjusted with respect to GAPDH transcript. For each gene, the highest expression value was considered 100% and transcripts of all pigs | | | | | | | | |
| #were adjusted relative to this pig. | | | | | | | | |

TABLE 7
EXPRESSION LEVELS (RT-PCR) OF DIFFERENT CANDIDATE
GENES IN MUSCLE FIBERS OF NORMAL AND AFFECTED PIGS

| | SEX | | STEATOSIS | | | P | | |
|---|---|---|---|---|---|---|---|---|
| GENE | M | F | Affected | Normal | SEM | Sex | Steatosis | S/ST |
| FGF-2 | 56.32 | 50.21 | 36.72 | 69.81 | 5.167 | 0.413 | 0.001 | 0.960 |
| EGF | 64.94 | 65.38 | 69.84 | 60.49 | 6.317 | 0.962 | 0.308 | 0.171 |
| IGF1 | 66.76 | 61.17 | 64.91 | 63.02 | 3.371 | 0.254 | 0.695 | 0.278 |
| IGF1R | 85.96 | 78.93 | 80.29 | 84.60 | 2.259 | 0.040 | 0.192 | 0.564 |
| IGF2 | 72.96 | 49.09 | 55.68 | 66.38 | 6.215 | 0.013 | 0.238 | 0.720 |
| IGF2R | 75.98 | 57.28 | 73.43 | 59.83 | 5.418 | 0.024 | 0.091 | 0.706 |
| PDGFα | 68.76 | 51.01 | 66.70 | 53.07 | 3.959 | 0.005 | 0.024 | 0.254 |
| PDGFβ | 69.38 | 63.33 | 57.01 | 75.70 | 3.983 | 0.295 | 0.003 | 0.230 |
| TGFα | 49.81 | 48.36 | 47.25 | 50.92 | 5.900 | 0.864 | 0.665 | 0.491 |
| aFGF | 67.98 | 57.86 | 64.64 | 61.21 | 8.418 | 0.406 | 0.776 | 0.809 |
| TGFβ | 54.31 | 51.18 | 46.97 | 58.52 | 4.912 | 0.657 | 0.112 | 0.009 |
| LPL | 64.66 | 66.60 | 60.68 | 70.59 | 3.155 | 0.669 | 0.038 | 0.090 |
| Legend: M, males; F, females; S/ST, Sex*Steatosis; SEM, standard deviation/√n; P, probability; significant when < 0.05 (shaded). Values in this table correspond to relative optical density that were adjusted with respect to GAPDH transcript. For each gene, the highest expression value was considered 100% and transcripts of all | | | | | | | | |
| #pigs were adjusted relative to this pig. | | | | | | | | |

TABLE 8
INTRAMUSCULAR LEVELS OF FGF-2, TGFA AND LEPTIN.

| | | | |
|---|---|---|---|
| normal | | | |
| females | normal males | affected females | affected males |

INTRAMUSCULAR FAT

| | | | | |
|---|---|---|---|---|
| FGF-2 | 34.20 | 37.54 | 68.71 | 38.05 |
| TGFα | 44.86 | 39.68 | 82.59 | 50.46 |
| Leptin | 49.13 | 48.54 | 79.17 | 58.46 |

MUSCLE FIBERS

| | | | | |
|---|---|---|---|---|
| TGFβ | 67.06 | 49.97 | 35.29 | 58.65 |

Values in this table correspond to relative optical density that were adjusted with respect to GAPDH transcript. For each gene, the highest expression value was considered 100% and transcripts of all pigs were adjusted relative to this pig.

### EXAMPLE II

### Identification of Molecular Markers

Experimental Method

Animal selection and sampling: 48 castrated commercial pigs from a same producer were used. These pigs were allocated according to a two-by-two factorial design in complete blocks with, as principal effects, the level of steatosis (24 pigs with steatosis levels ³4; 24 normal pigs) and the adiposity level (24 fat pigs with P₂≧22 mm between third and fourth ribs; 24 lean pigs with P₂≦19 mm). The animals were selected at the slaughterhouse the morning of the day of slaughter using an ultrasound machine. For each selected pig, blood was drawn just prior to their slaughter. At slaughter, the entire left semi-tendinosus muscle was taken, the fat trimmed off and then cut transversally in order to evaluate the steatosis level. Wrongly identified pigs were immediately replaced by new pigs on the same day of slaughter. The two hams, the loin, the flank, the liver and a sample of backfat were taken for further biochemical and genetic analysis.

Validation of results obtained: This part of the example has allowed us to confirm the results obtained in the preceding example. More precisely, we have performed the following analyses:

Measure of vitamin E: Vitamin E in the liver was performed by HPLC (high performance liquid chromatography) according to the protocol of Bieri et al. (1979, Am. J. Clin. Nutri. 32:2143-2149). First, the lipids were extracted using organic solvents (hexane or heptane) and the analysis of the tocopherols was done on a C18 column (inverse phase) which permits a fine separation of the different tocopherols.

Total carnitine levels: It was determined in plasma and in muscle, according to the radio-isotopic method developed by McGarry and Foster (197G, J. Lipid Res., 17:277-281).

All carcass and meat quality analyses, including pH at 45 minutes and ultimate pH was performed on all three studied muscles (semi-tendinosus, semi-membranosus and biceps femoris), and the loin and flank; allocation of the visual steatosis and marbling levels; determination of the percentage of dry matter of the loin, the flank and the three ham muscles; the percentage of lipids in the loin, the flank and the semi-tendinosus; measures of the backfat and muscle thickness as well as the muscle surface at the site of carcass classification (between the 3rd and 4th last ribs); water retention by the loin, the flank and the three ham muscles; the levels of glucose in the lost water; total protein content. The incidence of PSE meat was evaluated by measuring the color on the ventral side of the longissimus dorsi in the middle of the loin as well as on the three ham muscles studied. Digital images were taken of the transversal cuts of the studied muscles.

Measure of sub-cutaneous and intramuscular fatty acids by gas chromatography: The intra-muscular lipids (semi-tendinosus muscle) were extracted with chloroform-methanol, according to the method of Folch et al. (1957, J. Biol. Chem. 226:592-596). Total fatty acids was esterified according to the method of AOAC Official Method 991.39 (1995) and then analyzed by gas chromatography.

Expression levels of the bFGF gene (basic growth factor of fibroblasts) in intra-muscular fat and in muscle fibers: For these analyses, we begun by manually separating the muscle fibers from the intramuscular fat, under a binocular. This separation enabled us to evaluate the expression of bFGF specifically in intra-muscular fat and in muscle fibers. Once the separation has been completed, total RNA extracted and RT-PCR (Reverse Transcription-Polymerase Chain Reaction) analyses was performed for the bFGF gene. The RT-PCR analyses permitted us to quantify this gene's expression (i.e. the quantity of RNA expressed by this particular gene) and to verify if there are any differences between normal and affected pigs.

Comprehension of the mechanisms involved in the development of steatosis at the cellular and tissular level: This aspect of the example permitted us to point out the mechanism of the development of steatosis.

Measure of bound carnitine: In the preceding example, no significant difference was observed in the levels of total carnitine in muscle. However, a significant increase was observed in plasmatic carnitine in pigs affected by steatosis. These results gave us no information concerning the proportion of carnitine bound to fatty acids and free carnitine. This permitted us to verify if there is indeed a problem with the association of carnitine to long-chain fatty acids.

Determination of the levels of vitamin E in the muscles studied: In the previous example, we observed that steatosis-affected pigs accumulate more vitamin E in their liver than do normal pigs. It is therefore necessary to measure the levels of vitamin E in the ham muscles in order to verify if there is less vitamin E in affected muscles, which could entail a higher oxidative stress to these muscles.

Measure of the peroxydation levels of fatty acids: The decrease of certain fatty acids. (C15:1, C18:2 and C20:4) observed in the preceding example could be due to a higher peroxydation activity in affected muscles. In order to verify this, these levels of peroxydation in the semi-tendinosus muscle were measured according to the method of Witte et al. (1970, J. Food. Sci. 35:582-585).

Identification of new, easily measurable metabolic or genetic factors: This section permitted us to identify other factors, such as fatty acids, proteins or genes which t permit us to rapidly discriminate between affected and non-affected pigs, by way of simple fat tissue biopsies or blood samples.

Measure of the fatty acids present in red blood cells: This part of the example permitted us to identify if there are differences in the fatty acid profiles of affected pigs. The identification of differences in one or more fatty acids in red blood cells permitted us to use the blood of animals to determine their steatosis levels by simple gas chromatography analysis.

Identification of genes involved in the development of steatosis: To identify these genes, we have used a new molecular biology technique called “subtractive libraries”. This technique has permitted us to compare two populations of messenger RNA (expression levels of a gene) in order to obtain clones of genes that are expressed strongly in one population (steatosis-affected pigs) and weakly, or not at all, in the other (normal pigs) and vice-versa. In order to help us achieve this aspect of the example, we have used two kits commercialized by CLONTECH: “PCR-Select Differential Screening Kit” and “PCR-Select cDNA Subtraction Kit”. These analyses were performed on subcutaneous fat, intra-muscular fat and muscle fibers.

Results

Results of the second experiment are summarized in tables 9 to 16 respectively.

TABLE 9
CARNITINE ANALYSIS IN DIFFERENT TISSUES

| | | | | | | | P | | |
|---|---|---|---|---|---|---|---|---|---|
| | | Normal | | Affected | | | Adipo- | Stea- | |
| Variable | Tissue | Fat | Lean | Fat | Lean | SEM | sity | tosis | A/St |
| Carnitine (total) | Muscle | 58.11 | 57.12 | 62.06 | 55.20 | 3.35 | 0.217 | 0.747 | 0.354 |
| (nmoles/mg | | | | | | | | | |
| protein) | | | | | | | | | |
| Carnitine (free) | Muscle | 39.23 | 37.14 | 39.87 | 35.02 | 2.73 | 0.183 | 0.774 | 0.592 |
| (nmoles/mg | | | | | | | | | |
| protein) | | | | | | | | | |
| Carnitine (bound) | Muscle | 18.88 | 19.98 | 22.19 | 20.18 | 1.78 | 0.785 | 0.357 | |
| (nmoles/mg | | | | | | | | | |
| protein) | | | | | | | | | |
| Carnitine (total) | Plasma | 25.98 | 30.78 | 36.91 | 41.19 | 4.88 | 0.325 | 0.024 | 0.954 |
| (umoles/liter) | | | | | | | | | |
| Carnitine (free) | Plasma | 16.98 | 21.32 | 25.33 | 25.18 | 2.64 | 0.400 | 0.017 | 0.368 |
| (umoles/liter) | | | | | | | | | |
| Carnitine (bound) | Plasma | 9.00 | 9.47 | 11.58 | 16.01 | 2.56 | 0.312 | 0.063 | 0.412 |
| (umoles/liter) | | | | | | | | | |

legend: Fat, backfat ≧22 mm; Lean, backfat ≦19 mm; A/St, adiposity*steatosis; SEM, standard deviation/√n (n = 12); P, probability; significant when p < 0.05 (shaded).

TABLE 10
VITAMIN E ANALYSIS IN DIFFERENT TISSUES

| | | | | | | | P | | |
|---|---|---|---|---|---|---|---|---|---|
| | | Normal | | Affected | | | Adipo- | Stea- | |
| Variable | Tissue | Fat | Lean | Fat | Lean | SEM | sity | tosis | A/St |
| α-tocopherol | liver | 2.14 | 2.04 | 3.81 | 3.55 | 0.147 | 0.244 | 0.000* | 0.594 |
| (ug/g tissue) | | | | | | | | | |
| γ-tocopherol | liver | 0.17 | 0.17 | 0.15 | 0.17 | 0.018 | 0.399 | 0.516 | 0.525 |
| (ug/g tissue) | | | | | | | | | |
| α-tocopherol | muscle | 0.86 | 0.96 | 0.16 | 1.05 | 0.078 | 0.975 | 0.022 | 0.215 |
| (ug/g tissue) | | | | | | | | | |
| γ-tocopherol | muscle | 0.21 | 0.25 | 0.26 | 0.24 | 0.022 | 0.581 | 0.564 | 0.241 |
| (ug/g tissue) | | | | | | | | | |
| α-tocopherol | muscle | 1.02 | 1.01 | 0.44 | 1.17 | 0.135 | 0.012 | 0.140 | 0.012 |
| (ug/g tissue) | | | | | | | | | |
| γ-tocopherol | muscle | 0.20 | 0.22 | 0.12 | 0.21 | 0.020 | 0.015 | 0.047 | 0.115 |
| (ug/g tissue) | | | | | | | | | |

Legend: Fat, backfat ≧22 mm; Lean, backfat ≦19 mm; A/St, adiposity*steatosis; SEM, standard deviation/√n (n = 12); P, probability; significant when p < 0.05 (shaded). 0.000* ,p ≦ 0.00001.

TABLE 11
PEROXIDATION LEVELS OF INTRA-MUSCULAR FAT BY THE
THIOBARBITURIC ACID (TBA) METHOD
(WHITE ZONE OF THE SEMI-TENDINOSUS MUSCLE)

| | | | | | | P | | |
|---|---|---|---|---|---|---|---|---|
| | Normal | | Affected | | | Adipo- | Stea- | |
| Variable | Fat | Lean | Fat | Lean | SEM | sity | tosis | A/St |
| TBAJ0 | 0.144 | 0.155 | 0.128 | 0.179 | 0.018 | 0.051 | 0.924 | 0.180 |
| TBAJ4 | 0.177 | 0.212 | 0.217 | 0.241 | 0.022 | 0.151 | 0.123 | 0.845 |
| TBAJ9 | 0.201 | 0.221 | 0.318 | 0.288 | 0.391 | 0.891 | 0.015 | 0.489 |

TABLE 12
RED ZONE OF THE SEMI-TENDINOSUS MUSCLE

| | | | | | | P | | |
|---|---|---|---|---|---|---|---|---|
| | Normal | | Affected | | | Adipo- | Stea- | |
| Variable | Fat | Lean | Fat | Lean | SEM | sity | tosis | A/St |
| TBAJ0 | 0.242 | 0.220 | 0.164 | 0.188 | 0.032 | 0.743 | 0.147 | 0.254 |
| TBAJ4 | 0.270 | 0.289 | 0.280 | 0.265 | 0.027 | 0.742 | 0.907 | 0.796 |
| TBAJ9 | 0.295 | 0.289 | 0.342 | 0.340 | 0.033 | 0.791 | 0.076 | 0.836 |

Legend: Fat, backfat ≧22 mm; Lean, backfat ≦19 mm; A/St, adiposity*steatosis; SEM, standard deviation/√n (n = 12); TBA, thiobarbituric acid; J0, J4, J9, days 0, 4 and 9 respectively; P, probability; significant when p < 0.05 (shaded).

TABLE 13
EXPRESSION LEVELS (RT-PCR) OF bFGF IN NORMAL AND
STEATOSIS-AFFECTED PIGS

| | | | | | | P | | |
|---|---|---|---|---|---|---|---|---|
| | Normal | | Affected | | | Adipo- | Stea- | |
| Variable | Fat | Lean | Fat | Lean | SEM | sity | tosis | A/St |
| bFGF | 0.790 | 0.956 | 0.572 | 0.606 | 0.13 | 0.403 | 0.022 | 0.582 |
| muscle | | | | | | | | |
| fibers | | | | | | | | |
| (ng ratio) | | | | | | | | |
| bFGF intra- | 1.259 | 1.175 | 1.671 | 2.135 | 0.24 | 0.386 | 0.003 | 0.214 |
| muscular fat | | | | | | | | |
| (ng ratio) | | | | | | | | |
| Legend: Fat, backfat ≧22 mm; Lean, backfat ≦19 mm; A/St, adiposity*steatosis; SEM, standard deviation/√n (n = 12); P, probability; significant when p < 0.05 (shaded). The values in the table represent the quantity in ng calculated according to a standard curve. The relative values are standardized according to the pig | | | | | | | | |
| #with the highest level of mRNA expression. | | | | | | | | |

TABLE 14
ERYTHROCYTE FATTY ACID COMPOSITION (%)
IN NORMAL AND AFFECTED PIGS

| | | | | | | P | | |
|---|---|---|---|---|---|---|---|---|
| Fatty | Normal | | Affected | | | | Stea- | |
| acid | Fat | Lean | Fat | Lean | SEM | Adiposity | tosis | A/St |
| C14:0 | 0.22 | 0.23 | 0.21 | 0.22 | 0.012 | 0.374 | 0.651 | 0.830 |
| C15:0 | 0.16 | 0.17 | 0.13 | 0.14 | 0.015 | 0.444 | 0.062 | 0.640 |
| C16:0 | 21.05 | 20.95 | 20.66 | 20.99 | 0.382 | 0.772 | 0.657 | 0.583 |
| C16:1 | 0.56 | 0.60 | 0.55 | 0.55 | 0.022 | 0.361 | 0.227 | 0.449 |
| C17:0 | 0.78 | 0.76 | 0.66 | 0.75 | 0.040 | 0.447 | 0.132 | 0.163 |
| C17:1 | 0.18 | 0.18 | 0.18 | 0.19 | 0.013 | 0.868 | 0.937 | 0.847 |
| C18:0 | 16.46 | 17.18 | 15.92 | 16.67 | 0.339 | 0.041 | 0.138 | 0.962 |
| C18:1n9t | 0.97 | 0.99 | 0.93 | 0.96 | 0.026 | 0.306 | 0.227 | 0.889 |
| C18:1n9c | 21.46 | 20.87 | 19.70 | 21.63 | 0.906 | 0.472 | 0.594 | 0.181 |
| C18:1c11 | 0.87 | 0.92 | 0.80 | 0.86 | 0.021 | 0.010 | 0.003 | 0.647 |
| C18:2n6c | 31.38 | 30.91 | 34.31 | 30.73 | 1.306 | 0.137 | 0.311 | 0.251 |
| C18:3n3 | 0.31 | 0.34 | 0.36 | 0.36 | 0.027 | 0.607 | 0.221 | 0.648 |
| C20:0 | 0.13 | 0.11 | 0.11 | 0.13 | 0.007 | 0.526 | 0.838 | 0.023 |
| C20:1 | 0.22 | 0.20 | 0.22 | 0.19 | 0.017 | 0.179 | 0.556 | 0.697 |
| C20:2 | 0.31 | 0.30 | 0.26 | 0.29 | 0.009 | 0.310 | 0.003 | 0.016 |
| C20:3n6 | 0.51 | 0.46 | 0.42 | 0.46 | 0.025 | 0.995 | 0.103 | 0.114 |
| C20:4n6 | 4.31 | 4.70 | 4.47 | 4.74 | 0.187 | 0.088 | 0.610 | 0.765 |
| C22:0 | 0.12 | 0.13 | 0.12 | 0.14 | 0.014 | 0.285 | 0.914 | 0.757 |

Legend: Fat, backfat ≧22 mm; Lean, backfat ≦19 mm; A/St, adiposity*steatosis; SEM, standard deviation/√n (n = 12); P, probability; significant when p < 0.05 (shaded).

TABLE 15
BACKFAT FATTY ACID COMPOSITION (%) IN NORMAL AND
AFFECTED PIGS

| | | | | | | P | | |
|---|---|---|---|---|---|---|---|---|
| | Normal | | Affected | | | Adi- | Stea- | |
| Fatty acid | Fat | Lean | Fat | Lean | SEM | posity | tosis | A/St |
| C14:0 | 1.65 | 1.55 | 1.60 | 1.51 | 0.033 | 0.008 | 0.227 | 0.861 |
| C16:0 | 27.73 | 25.86 | 27.62 | 25.92 | 0.324 | 0.000* | 0.954 | 0.792 |
| C16:1 | 2.63 | 2.62 | 2.41 | 2.41 | 0.075 | 0.936 | 0.009 | 0.911 |
| C17:0 | 0.28 | 0.29 | 0.25 | 0.29 | 0.013 | 0.101 | 0.167 | 0.452 |
| C17:1 | 0.28 | 0.29 | 0.23 | 0.27 | 0.013 | 0.073 | 0.035 | 0.289 |
| C18:0 | 12.59 | 12.00 | 14.60 | 13.78 | 0.219 | 0.003 | 0.000* | 0.596 |
| C18:1n9t | 0.63 | 0.65 | 0.64 | 0.70 | 0.033 | 0.249 | 0.371 | 0.671 |
| C18:1n9c | 29.57 | 29.10 | 28.81 | 28.95 | 0.342 | 0.644 | 0.196 | 0.386 |
| C18:1c11 | 1.73 | 1.74 | 1.61 | 1.65 | 0.034 | 0.525 | 0.004 | 0.677 |
| C18:2n6t | 0.13 | 0.14 | 0.12 | 0.13 | 0.003 | 0.005 | 0.029 | 0.211 |
| C18:2n6c | 19.39 | 22.23 | 18.81 | 21.00 | 0.425 | 0.000* | 0.044 | 0.457 |
| C18:3n3 | 0.79 | 0.91 | 0.73 | 0.84 | 0.024 | 0.00002 | 0.016 | 0.883 |
| C20:0 | 0.19 | 0.15 | 0.21 | 0.18 | 0.011 | 0.005 | 0.050 | 0.662 |
| C20:1 | 1.13 | 1.05 | 1.12 | 1.04 | 0.039 | 0.050 | 0.822 | 0.963 |
| C20:2 | 0.64 | 0.69 | 0.62 | 0.66 | 0.014 | 0.003 | 0.054 | 0.872 |
| C20:3n6 | 0.13 | 0.12 | 0.11 | 0.11 | 0.009 | 0.664 | 0.150 | 0.419 |
| C20:3n3 | 0.13 | 0.14 | 0.12 | 0.12 | 0.004 | 0.043 | 0.010 | 0.252 |
| C20:4n6 | 0.21 | 0.26 | 0.21 | 0.25 | 0.009 | 0.00003 | 0.527 | 0.479 |
| C21:0 | 0.18 | 0.22 | 0.17 | 0.19 | 0.009 | 0.005 | 0.017 | 0.408 |

Legend: Fat, backfat ≧22 mm; Lean, backfat ≦19 mm; A/St, adiposity*steatosis; SEM, standard deviation/√n (n = 12); P, probability; significant when p < 0.05 (shaded); 0.000*, p ≦ 0.00001.

TABLE 16
MUSCLE FATTY ACID COMPOSITION (%)
IN NORMAL AND AFFECTED PIGS

| | | | | | | P | | |
|---|---|---|---|---|---|---|---|---|
| | Normal | | Affected | | | Adi- | Stea- | |
| Fatty acid | Fat | Lean | Fat | Lean | SEM | posity | tosis | A/St |
| C14:0 | 1.47 | 1.41 | 1.67 | 1.47 | 0.040 | 0.002 | 0.003 | 0.109 |
| C16:0 | 28.76 | 27.74 | 30.70 | 28.55 | 0.313 | 0.00001 | 0.0001 | 0.087 |
| C16:1 | 3.10 | 3.10 | 3.23 | 2.87 | 0.144 | 0.233 | 0.753 | 0.227 |
| C17:0 | 0.21 | 0.20 | 0.18 | 0.21 | 0.011 | 0.544 | 0.259 | 0.183 |
| C17:1 | 0.23 | 0.24 | 0.20 | 0.23 | 0.013 | 0.087 | 0.133 | 0.337 |
| C18:0 | 14.41 | 13.70 | 15.18 | 14.81 | 0.315 | 0.101 | 0.006 | 0.595 |
| C18:1n9t | 0.77 | 0.84 | 0.85 | 0.84 | 0.041 | 0.487 | 0.346 | 0.385 |
| C18:1n9c | 27.36 | 27.15 | 28.46 | 28.27 | 0.570 | 0.734 | 0.063 | 0.992 |
| C18:1c11 | 2.03 | 2.11 | 1.97 | 1.95 | 0.063 | 0.657 | 0.093 | 0.459 |
| C18:2n6c | 17.82 | 19.22 | 14.40 | 17.23 | 0.565 | 0.001 | 0.00003 | 0.226 |
| C18:3n3 | 0.55 | 0.59 | 0.51 | 0.56 | 0.028 | 0.135 | 0.317 | 0.895 |
| C20:0 | 0.15 | 0.12 | 0.18 | 0.16 | 0.011 | 0.024 | 0.002 | 0.976 |
| C20:1 | 0.84 | 0.86 | 0.89 | 0.89 | 0.032 | 0.751 | 0.223 | 0.713 |
| C20:2 | 0.42 | 0.46 | 9.40 | 0.45 | 0.015 | 0.004 | 0.323 | 0.606 |
| C20:3n6 | 0.25 | 0.28 | 0.16 | 0.20 | 0.018 | 0.058 | 0.00004 | 0.780 |
| C20:4n6 | 1.52 | 1.87 | 0.90 | 1.20 | 0.122 | 0.013 | 0.00001 | 0.849 |
| C21:0 | 0.11 | 0.11 | 0.11 | 0.11 | 0.009 | 0.941 | 0.840 | 0.682 |

Legend: Fat, backfat ≧22 mm; Lean, backfat ≦19 mm; A/St, adiposity*steatosis; SEM, standard deviation/√n (n = 12); P, probability; significant when p < 0.05 (shaded).

In conclusion, the present experiment demonstrates clearly that several MSMF are correlated with the steatotic state in pigs. It has been determined that the muscular superoxide dismutase, and hepatic Vitamin E are correlated with the muscular steatosis. In addition, it can be seen from the present results that fatty acids have a direct relation with the muscular steatosis, as well in sub-cutaneous as muscular samples. Also, from the RT-PCR discrimination performed in fat or muscular samples, it was observed that EGF, IGF1R, IGF2, aFGF, FGF-2, TGFα, PDGFα, PDGFβ, LPL, and the Leptin are each one good markers in determining the steatosis status of animals. Most particularly, the present invention shows that amplification of selected MSMF, it is to say the Leptin, FGF-2 and IGF1R are particularly accurates for identifying differential genetic expression in diagnosing the steatosis. The FGF-2 allows discrimination of steatotic pigs in 91.67 percent. Combination of factors makes possible to select non-steatotic from steatotic individuals in closed to 99 percents of the cases.

While the invention has been described in connection with specific embodiments thereof, it has been understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features herein before set forth, and as follows in the scope of the appended claims.

What is claimed is:

1. A method for prognosis or diagnosis of muscular steatosis based on the level of muscular steatosis-modulating factor (MSMF) in a human or animal, which comprises the steps of:
a) measuring level of at least one MSMF in a biological sample of said human or animal, and
b) comparing the level of MSMF measured in the biological sample of said human or animal with the level of MSMF measured in a biological sample of a healthy human or animal, wherein a difference indicates predisposition or occurrence of steatosis.

2. A method according to claim 1, wherein said animal is selected from the group consisting of mammal and avian.

3. A method according to claim 1, wherein said animal is selected from the group consisting of porcine, bovine, ovine, caprine, chicken, turkey, horse, goat, canine, and feline.

4. A method according to claim 1, wherein said measuring of step a) is performed by identifying differential expression of MSMF gene.

5. A method according to claim 4, wherein said MSMF gene comprises a nucleic acid sequence selected from the group consisting of SEQ ID NO:1 to SEQ ID NO:305 or a fragment thereof.

6. A method according to claim 1, wherein said MSMF is selected from the group consisting of growth hormone, growth factor, cytokine, growth factor receptor, growth hormone receptor, cytokine receptor, and lipid.

7. A method according to claim 1, wherein said MSMF is IGF1, IGF2, αFGF, FGF-2, ADRP, IGF1R, PDGFα, TGFβ, TGFα, LPL, EGF, PDGFβ, Leptin, superoxide dismutase, carnitine, creatine kinase, a vitamin, or a combination thereof.

8. A method according to claim 1, wherein said biological sample is blood, serum, plasma, a biopsy, fat, saliva, feces, or urine.

9. A method according to claim 1, wherein said measuring MSMF of step a) consists of measuring the level of at least one peptide, precursor, metabolite, or a messenger RNA of MSMF.

10. A method according to claim 9, wherein said messenger RNA is complementary to a nucleic acid sequence selected from the group consisting of SEQ ID NO:1 to SEQ ID NO:305 or a fragment thereof.

11. A method according to claim 9, wherein said messenger RNA is corresponding to a nucleic acid sequence selected from the group consisting of SEQ ID NO:1 to SEQ ID NO:305 or a fragment thereof.

12. A method for the treatment of muscular steatosis in a human or an animal, which comprises regulating MSMF level substantially equivalent to that of a healthy human or a healthy animal.

13. A method according to claim 12, wherein said regulation is performed by administrating an agonist of MSMF, an antagonist of MSMF, or a combination thereof.

14. A method according to claim 13, wherein said agonist of MSMF is a MSMF.

15. A method according to claim 13, wherein said agonist of MSMF is a recombinant MSMF, a precursor of MSMF, a non-mature MSMF, an analog of MSMF, a purified MSMF, or a physiologically active fragment of at least one MSMF.

16. A method according to claim 13, wherein said agonist of MSMF is an abzyme.

17. A method according to claim 13, wherein said antagonist of MSMF is an inhibitor of MSMF.

18. A method according to claim 13, wherein said antagonist of MSMF is an abzyme.

19. A method according to claim 13, wherein said antagonist of MSMF is selected from the group consisting of an antibody, an anti-MSMF messenger RNA, a MSMF RNA ligand, a MSMF-specific antisense primer, an anti-MSMF receptor, and a mutant MSMF.

20. A method according to claim 19, wherein said anti-MSMF messenger RNA is complementary to a nucleic acid sequence selected from the group consisting of SEQ ID NO:1 to SEQ ID NO:305 or a fragment thereof.

21. A method according to claim 19, wherein said anti-MSMF messenger RNA is corresponding to a nucleic acid sequence selected from the group consisting of SEQ ID NO:1 to SEQ ID NO:305 or a fragment thereof.

22. A method according to claim 13, wherein said agonist of MSMF, antagonist of MSMF, or combination thereof is administered by introducing at least one expression vector into the human or the animal.

23. A method according to claim 22, wherein the expression vector is introduced into at least one cell, and said cell is introduced into the human or the animal.

24. A method according to claim 13, wherein the agonist of MSMF or the antagonist of MSMF is administered systemically, orally, or intravenously, using an implant or a slow delivery system.

25. A method for causing muscular steatosis in an animal for increasing fat content, which comprises the step of administrating to said animal a sufficient amount of at least one agonist of MSMF, an antagonist of MSMF, or a combination thereof, for deregulating MSMF in said animal to a level different from the level of MSMF of a healthy animal.

26. A method according to claim 25, wherein said agonist of MSMF is a MSMF.

27. A method according to claim 25, wherein said agonist of MSMF is selected from the group consisting of recombinant MSMF, precursor of MSMF, non-mature MSMF, analog of MSMF, purified MSMF, and a physiologically active fragment of a MSMF.

28. A method according to claim 27, wherein said agonist of MSMF is an abzyme.

29. A method according to claim 25, wherein said antagonist of MSMF is a MSMF.

30. A method according to claim 25, wherein said antagonist of MSMF is an abzyme.

31. A method according to claim 25, wherein said antagonist is selected from the group consisting of an antibody, an anti-MSMF messenger RNA, a MSMF RNA ligand, a MSMF-specific antisense primer, an anti-MSMF receptor, a synthetic antisenses, a natural antisenses, and a mutant MSMF.

32. A method according to claim 31, wherein said anti-MSMF messenger RNA is complementary to a nucleic acid sequence selected from the group consisting of SEQ ID NO:1 to SEQ ID NO:305 or a fragment thereof.

33. A method according to claim 31, wherein said anti-MSMF messenger RNA is corresponding to a nucleic acid sequence selected from the group consisting of SEQ ID NO:1 to SEQ ID NO:305 or a fragment thereof.

34. A method according to claim 25, wherein said agonist, antagonist, or combination thereof is administered by introducing at least one expression vector into the animal.

35. A method according to claim 34, wherein said expression vector is introduced into at least one cell, and said cell is introduced into said animal.

36. A method according to claim 25, wherein an agonist or antagonist is administered systemically, orally, or intravenously, using an implant or a slow delivery system.

37. A method according to claim 35, wherein said muscular steatosis is caused in said animal by administrating an agonist of MSMF, an antagonist of MSMF, or a combination thereof.

38. A compound of the group of MSMF for the treatment of muscular steatosis in a human or an animal patient.

39. A compound according to claim 38, which is selected from the group consisting of an agonist of MSMF and an antagonist of MSMF, or a combination thereof.

40. A compound according to claim 39, wherein said agonist of MSMF is a MSMF.

41. A compound according to claim 39, wherein said agonist is a recombinant MSMF, a precursor of MSMF, a non-mature MSMF, an analog of MSMF, a purified MSMF, or a physiologically active fragment of a MSMF.

42. A compound according to claim 39, wherein said agonist of MSMF is an abzyme.

43. A compound according to claim 39, wherein said antagonist of MSMF is a MSMF.

44. A compound according to claim 39, wherein said antagonist of MSMF is an abzyme.

45. A compound according to claim 39, wherein said antagonist of MSMF is selected from the group consisting of an antibody, an anti-MSMF messenger RNA, a MSMF RNA ligand, a MSMF-specific antisense primer, an anti-MSMF receptor, and a mutant MSMF.

46. A method according to claim 45, wherein said anti-MSMF messenger RNA is complementary to a nucleic acid sequence selected from the group consisting of SEQ ID NO:1 to SEQ ID NO:305 or a fragment thereof.

47. A method according to claim 45, wherein said anti-MSMF messenger RNA is corresponding to a nucleic acid sequence selected from the group consisting of SEQ ID NO:1 to SEQ ID NO:305 or a fragment thereof.

48. A compound according to claim 39, wherein said agonist of MSMF, antagonist of MSMF or combination thereof is administered by introducing at least one expression vector into the human or the animal patient.

49. A compound according to claim 48, wherein said expression vector is introduced into at least one cell, and said cell is introduced into said human or said animal patient.

50. A compound according to claim 39, wherein said agonist of MSMF or antagonist of MSMF is administered systemically, orally, or intravenously, using an implant or a slow delivery system.

51. A compound of the group of MSMF for causing muscular steatosis in an animal.

52. A compound according to claim 51, which is selected from the group consisting of an agonist of MSMF and an antagonist of MSMF, or a combination thereof.

53. A compound according to claim 51, wherein said agonist of MSMF is a MSMF.

54. A compound according to claim 52, wherein said agonist of MSMF is a recombinant MSMF, a precursor of MSMF, a non-mature MSMF, an analog of MSMF, a purified MSMF, or a physiologically active fragment of a MSMF.

55. A compound according to claim 52, wherein said agonist of MSMF is an abzyme.

56. A compound according to claim 52, wherein said antagonist of MSMF is a MSMF.

57. A compound according to claim 52, wherein said antagonist of MSMF is an abzyme.

58. A compound according to claim 52, wherein said antagonist of MSMF is selected from the group consisting of an antibody, an anti-MSMF messenger RNA, a MSMF RNA ligand, a MSMF-specific antisense primer, an anti-MSMF receptor, and a mutant MSMF.

59. A compound according to claim 58, wherein said anti-MSMF messenger RNA is complementary to a nucleic acid sequence selected from the group consisting of SEQ ID NO:1 to SEQ ID NO:305 or a fragment thereof.

60. A compound according to claim 58, wherein said anti-MSMF messenger RNA is corresponding to a nucleic acid sequence selected from the group consisting of SEQ ID NO:1 to SEQ ID NO:305 or a fragment thereof.

61. A compound according to claim 52, wherein said agonist of MSMF, antagonist of MSMF or combination thereof is administered by introducing at least one expression vector into the animal.

62. A compound according to claim 61, wherein said expression vector is introduced into at least one cell, and said cell is introduced into said animal.

63. A compound according to claim 52, wherein said agonist of MSMF or antagonist of MSMF is administered systemically, orally, or intravenously, using an implant or a slow delivery system.

64. Use of a compound as defined in any one of claims 38 to 50 for treating muscular steatosis.

65. Use of a compound as defined in any one of claims 51 to 63 for causing muscular steatosis.

66. Use of a compound of the group of MSMF in the manufacture of a medicament for treating muscular steatosis.

67. Use of a compound as defined in any one of claims 38 to 50 in the manufacture of a medicament for treating muscular steatosis.

68. Use of a compound of the group of MSMF in the manufacture of a medicament for causing muscular steatosis.

69. Use of a compound as defined in any one of claims 51 to 63 in the manufacture of a medicament for inducing muscular steatosis.

70. A pharmaceutical composition comprising a compound as defined in any one of claims 38 to 50 in association with a pharmaceutical acceptable carrier.

71. A pharmaceutical composition for use in the treatment of muscular steatosis comprising a therapeutically acceptable and effective amount of a compound of the group of MSMF in association with a pharmaceutically acceptable carrier.

72. A pharmaceutical composition for use in causing muscular steatosis comprising a therapeutically acceptable and effective amount of a compound of the group of MSMF in association with a pharmaceutically acceptable carrier.
